SEQUENCE LISTING (1 sequence)

SEQ ID NO: 1 - DNA, 39 bp, Synthetic construct
  source 1..39: mol_type = other DNA
ttactcttctttttccgagccggacgactcttatttccc

(19) United States
(12) Patent Application Publication
(10) Pub. No.: US 2026/0317797 A1
(43) Pub. Date: Oct. 15, 2026

(54) IN VITRO TRANSCRIPTION METHOD

(71) Applicant: TriLink BioTechnologies, LLC, San Diego, CA (US)

(72) Inventors: Andrew UJITA, San Diego, CA (US); Anthony TRUONG, San Diego, CA (US); Jordana HENDERSON, San Diego, CA (US); Michael HOUSTON, San Diego, CA (US)

(73) Assignee: TriLink BioTechnologies, LLC, San Diego, CA (US)

(21) Appl. No.: 19/481,047

(22) PCT Filed: May 5, 2023
(86) PCT No.: PCT/US2023/066711

§ 371 Date: Nov. 3, 2025

(51) Int. Cl. C12P 19/34; C12N 9/12; C12N 9/16

(52) U.S. Cl. CPC C12P 19/34; C12N 9/1247; C12N 9/16; C12Y 207/07006; C12Y 301/21001

(57) ABSTRACT

Provided herein are methods for in vitro transcription of messenger RNA (mRNA). The methods are used for synthesizing capped messenger RNA (mRNA) transcripts that are substantially free of impurities such as DNA, protein, double-stranded RNA and/or incomplete mRNA transcripts.

### FIELD

The present application relates to in vitro methods for generating capped messenger RNAs for a variety of research and therapeutic applications.

### BACKGROUND

In vitro transcribed mRNA molecules are useful in numerous biological research and clinical applications. For example, in vitro transcribed mRNAs have numerous in vivo applications, such as replacement therapy, where mRNA encoding a therapeutic protein is administered to the patient; vaccination, where mRNA encoding specific antigen(s) is administered to elicit protective immunity in a patient; or cell therapy, where mRNA is transfected into cells ex vivo to alter cell phenotype or function prior to delivery of these altered cells to a patient. These applications require administration of purified, capped mRNAs that elicit a therapeutic effect, while minimizing the innate immune response caused by impurities typically associated with in vitro transcribed mRNAs. Therefore, methods for synthesizing capped mRNAs with reduced impurities, such as, for example, double stranded RNA, DNA or incomplete mRNA transcripts, and increased transcription yield, are necessary.

### SUMMARY

This summary is a high-level overview of various aspects of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification, any or all drawings, and each claim.

Provided herein is an in vitro method for synthesizing capped messenger RNA (mRNA) transcripts comprising: (a) forming a reaction mixture comprising: (i) 6.0 mM to 25 mM initiating capped oligonucleotide primer or a salt thereof (ii) 0.020 μg/μl to 0.1 μg/μl DNA template; (iii) nucleoside triphosphates (NTPs), including 3 mM to 7 mM ATP; 3 mM to 7 mM CTP; 3 mM to 7 mM GTP; and 3 mM to 7 mM UTP; (iv) 1× Transcription Buffer; (v) 10 U/μL to 18 U/μL RNA polymerase; and (vi) DNase and RNase free water, wherein the reaction mixture has a pH of 6.0 to 8.0; and (b) incubating the reaction mixture, for 2-3 hours, under conditions that allow transcription of the DNA template to obtain a solution comprising capped mRNA transcripts.

In embodiments, 10× Transcription Buffer comprises (a) about 400 mM Tris-HCl (pH 7.5); (b) about 100 mM dithiothreitol (DTT); (c) about 20 mM spermidine; (d) about 160 mM magnesium chloride; (e) about 150 mM hydrochloric acid; (f) DNase and RNase free water; and wherein the 10× Transcription Buffer has a pH of about 1.5.

In embodiments, the method further comprises incubating the solution comprising the capped mRNA transcripts with a Spike-in mix: (c) incubating the solution comprising capped mRNA transcripts with: (i) nucleoside triphosphates (NTPs), including ATP, CTP, GTP, and UTP, each with a concentration of 3 mM to 8 mM in a Final Reaction Mixture; and (ii) 10× Transcription Buffer, with a concentration of 1× Transcription Buffer in a Final Reaction Mixture. In embodiments, the incubation is performed at 37° C. for 1 to 3 hours. In embodiments, the incubation is performed at 37° C. for 2 hours.

In embodiments, the method further comprises incubating the solution comprising the capped mRNA transcripts or the Final Reaction Mixture with: (i) a solution of CaCl₂) with a concentration of 0.5 mM to 5 mM in the reaction mixture or the Final Reaction Mixture; and (ii) DNase I. In embodiments, the incubation is performed at 37° C. for 10 to 60 minutes. In embodiments, the incubation is performed at 37° C. for 20 minutes. In embodiments, the method further comprises subjecting the DNase treated reaction mixture to proteinase K treatment.

In embodiments, the method further comprises one or more purification steps to purify the capped mRNA transcripts. In embodiments, the one or more purification steps comprise one or more tangential flow filtration steps. In embodiments, the one or more purification steps comprise one or more oligo dT purification steps. In embodiments, the one or more purification steps comprise one or more lithium chloride (LiCl) precipitation. In embodiments, the one or more purification steps comprise one or more RNeasy purification. In embodiments, the one or more purification steps yield purified, capped mRNA transcripts that are substantially free of impurities.

Also provided herein is an in vitro method for synthesizing capped messenger RNA (mRNA) transcripts comprising: (a) forming a reaction mixture comprising: (i) 6.0 mM to 25 mM initiating capped oligonucleotide primer or a salt thereof; (ii) 0.020 μg/μl to 0.1 μg/μl DNA template; (iii) nucleoside triphosphates (NTPs), including 3 mM to 7 mM ATP; 3 mM to 7 mM CTP; 3 mM to 7 mM GTP; and 3 mM to 7 mM UTP; (iv) 1× Transcription Buffer; (v) 10 U/μL to 18 U/μL RNA polymerase; and (vi) DNase and RNase free water, wherein the reaction mixture has a pH of 6.0 to 8.0; (b) incubating the reaction mixture, for 2-3 hours, under conditions that allow transcription of the DNA template to obtain a solution comprising capped mRNA transcripts; (c) optionally incubating the solution comprising capped mRNA transcripts with a Spike-in mix: (i) nucleoside triphosphates (NTPs), including ATP, CTP, GTP, and UTP, each with a concentration of 3 mM to 8 mM in a Final Reaction Mixture; and (ii) 10× Transcription Buffer, with a concentration of 1× Transcription Buffer in a Final Reaction Mixture, wherein the incubation is performed at 37° C. for 1 to 3 hours; and (d) incubating the solution comprising the capped mRNA transcripts or the Final Reaction Mixture with: (i) a solution of CaCl₂) with a concentration of 0.5 mM to 5 mM in the reaction mixture or the Final Reaction Mixture; and (ii) DNase I, wherein the incubation is performed at 37° C. for 10 to 60 minutes. In embodiments, the DNAse I incubation is performed at 37° C. for 20 minutes.

In embodiments, the method further comprises subjecting the DNase treated reaction mixture to one or more purification steps. In embodiments, the one or more purification steps comprise oligo dT purification. In embodiments, the one or more purification steps comprise tangential flow filtration.

In embodiments, the DNase treated reaction mixture is subjected to oligo dT purification to obtain oligo dT purified mRNA transcripts. In embodiments, the method further comprises subjecting the oligo dT purified mRNA transcripts to tangential flow filtration to obtain purified, capped mRNA transcripts.

In embodiments, the method further comprises: (e) subjecting the DNase-treated reaction mixture to tangential flow filtration to obtain a first sample comprising purified, capped mRNA transcripts; (f) subjecting the first sample from step (e) to oligo dT purification to obtain a second sample comprising purified, capped mRNA transcripts; and (g) subjecting the second sample from step (f) to a second tangential flow filtration step to obtain a third sample comprising purified, capped mRNA transcripts. In embodiments, the first, second, and/or third sample comprising purified, capped mRNA transcripts are substantially free of impurities.

In embodiments, the initiating capped oligonucleotide primer is a trinucleotide. In embodiments, the initiating capped oligonucleotide primer comprises a 5′ to 5′ triphosphate bridge.

In embodiments, the initiating capped oligonucleotide primer is selected from ^{m7}Gppp^{m6}A_{2′Ome}pG or a salt thereof and ^{m7}G_{3′Ome}ppp^{m6} A_{2′Ome}pG or a salt thereof.

In embodiments, ^{m7}Gppp^{m6} A_{2′Omep}G has the following structure:

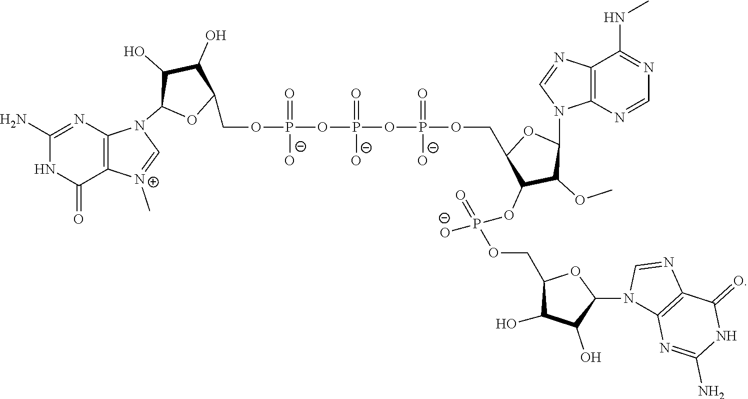

In embodiments, ^{m7}G_{3′Ome}ppp^{m6} A_{2′Ome}pG has the following structure:

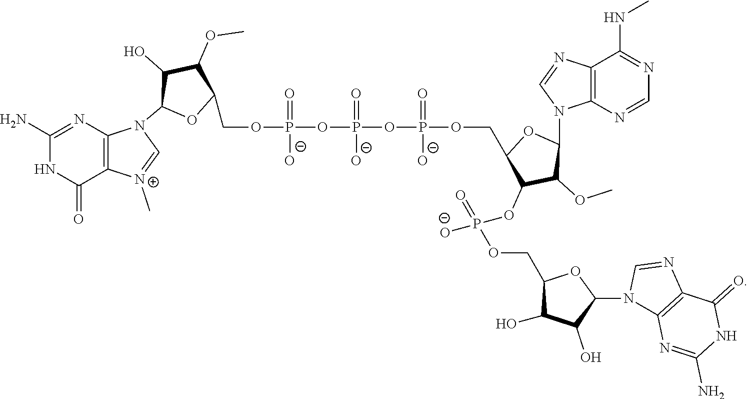

In embodiments, one or more of the NTPs used in the method is a modified NTP.

In embodiments, the reaction mixture further comprises one or more of an RNase inhibitor and/or an inorganic pyrophosphatase. In embodiments, the concentration of the RNase inhibitor is 1 U/μl. In embodiments, the concentration of the inorganic pyrophosphatase is 0.002 U/μl.

In embodiments, the reaction mixture is incubated at 34° C. to 44° C. for greater than one hour, for example, for 2 to 3 hours. In embodiments, the concentration of each NTP in the reaction mixture is 3 mM to 7 mM.

In embodiments, the concentration of Mg²⁺ in the reaction mixture is 15 mM to 17 mM. For example, the concentration of Mg²⁺ in the reaction mixture can be 16 mM.

In embodiments, the concentration of the RNA polymerase is 13 U/μL to 17 U/μL. In embodiments, the RNA polymerase is T7 RNA polymerase.

In embodiments, the pH of the reaction the reaction mixture is 6.6 to 7.0. In embodiments, the pH of the reaction buffer added to the reaction mixture is 6.8.

In embodiments, greater than 95% (e.g., 96%, 97%, 98%, or 99%) of the synthesized RNA transcripts are capped mRNA transcripts. In embodiments, less than 1% of the mass of the synthesized RNA transcripts is double stranded RNA. In embodiments, less than 0.0005% of the nucleic acid mass in a sample of synthesized RNA transcripts is DNA.

In embodiments, the method yields 7 to 13 mg/mL of mRNA transcripts. In embodiments, the capped RNA transcripts are between 0.3 kb to 10 kb in length. In embodiments, the DNA template is a linearized plasmid or a PCR product.

### DESCRIPTION OF THE FIGURES

The present application includes the following figures. The figures are intended to illustrate certain aspects and/or features of the methods described herein, and to supplement any description(s) of the compositions and methods. The figures do not limit the scope of the methods, unless the written description expressly indicates that such is the case.

FIG. 1A is a bar graph showing yields of mRNA transcripts prepared using Buffer 1 in vitro transcription (IVT) method—using CleanCap analogs m6A (^{m7}Gppp^{m6}A_{2′Ome}pG) or 3′OMe m6A (^{m7}G_{3′Ome}ppp^{m6} A_{2′Ome}pG), and firefly luciferase (Fluc) transcription template.

FIG. 1B is a bar graph showing capping efficiency of mRNA transcripts prepared using Buffer 1 in vitro transcription (IVT) method—using CleanCap analogs m6A (^{m7}Gppp^{m6} A_{2′Ome}pG) or 3′OMe m6A (^{m7}G_{3′Ome}ppp^{m6} A_{2′Ome}pG), and firefly luciferase (Fluc) transcription template.

FIG. 2 is a bar graph comparing yields of mRNA transcripts prepared using Buffer 1 IVT method vs Comparative Buffer IVT method-using CleanCap analog 3′OMe m6A (^{m7}G_{3′Ome}ppp^{m6} A_{2′Ome}pG) and firefly luciferase (Fluc) transcription template. The yields of mRNA transcripts prepared using original IVT method “Fixed” and new IVT method “Spiked”, described in Example 2, are also compared. The “Spiked” IVT method involves addition and further incubation of additional NTPs and appropriate Buffer, two hours after initial IVT reaction.

FIG. 3 is a bar graph comparing capping efficiency of mRNA transcripts prepared using Buffer 1 IVT method vs Comparative Buffer IVT method—using CleanCap analog 3′OMe m6A (^{m7}G_{3′Ome}ppp^{m6} A_{2′Ome}pG) and firefly luciferase (Fluc) transcription template. The capping efficiencies of mRNA transcripts prepared using original IVT method “Fixed” and new IVT method “Spiked”, described in Example 2, are also compared. The “Spiked” IVT method involves addition and further incubation of additional NTPs and appropriate Buffer, two hours after initial IVT reaction.

FIG. 4 is a bar graph comparing the amounts of unwanted byproduct, dsRNA, formed using Buffer 1 IVT method vs Comparative Buffer IVT method-using CleanCap analog 3′OMe m6A (^{m7}G_{3′Ome}ppp^{m6} A_{2′Ome}pG) and firefly luciferase (Fluc) transcription template. The amounts of unwanted byproduct, dsRNA, prepared using original IVT method “Fixed” and new IVT method “Spiked”, described in Example 2, are also compared. The “Spiked” IVT method involves addition and further incubation of additional NTPs and appropriate Buffer, two hours after initial IVT reaction.

### DETAILED DESCRIPTION

### Definitions

The following description recites various examples of the present methods. No particular example is intended to define the scope of the methods. Rather, these are non-limiting, exemplary methods. The description is to be read from the perspective of one of ordinary skill in the art; therefore, information well known to the skilled artisan is not necessarily included.

Unless defined otherwise, all technical and scientific terms used herein have the same meaning as commonly understood by one of ordinary skill in the art to which this disclosure belongs. All patents, patent applications and publications referred to throughout the disclosure herein are incorporated by reference in their entirety.

As used in the specification and the appended claims, the singular forms “a,” “an,” and “the” include plural referents unless the context clearly dictates otherwise. For example, reference to “a transcript” or “the transcript” may include a plurality of transcripts.

The use of any and all examples or exemplary language (e.g., “such as”) provided herein, is intended merely to better illustrate the invention and does not pose a limitation on the scope of the invention unless otherwise claimed.

The terms “may,” “may be,” “can,” and “can be,” and related terms are intended to convey that the subject matter involved is optional (that is, the subject matter is present in some examples and is not present in other examples), not a reference to a capability of the subject matter or to a probability, unless the context clearly indicates otherwise.

As used herein, the term “about” means a range of values including the specified value, which a person of ordinary skill in the art would consider reasonably similar to the specified value. In embodiments, “about” refers to a value or composition that is within an acceptable error range for the particular value or composition as determined by one of ordinary skill in the art, which will depend in part on how the value or composition is measured or determined, i.e., the limitations of the measurement system. In embodiments, “about” means within a standard deviation using measurements generally acceptable in the art. In embodiments, “about” means a range extending to +/−10% or more of the specified value, depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “approximately” should be assumed to be within an acceptable error range for that particular value or composition. In some embodiments, “about” encompasses variation within 10%, 5%, 2%, 1%, or 0.5% of a stated value. In embodiments, “about” includes the specified value.

The terms “optional” and “optionally” mean that the subsequently described event, circumstance, or material may or may not occur or be present, and that the description includes instances where the event, circumstance, or material occurs or is present as well as instances where it does not occur or is not present.

The use herein of the terms “including,” “comprising,” “containing,” or “having,” and variations thereof, is meant to encompass the elements listed thereafter and equivalents thereof as well as additional elements. Embodiments recited as “including,” “comprising,” or “having” certain elements are also contemplated as “consisting essentially of” and “consisting of” those certain elements.

It is understood the use of the alternative (e.g., “or”) herein is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

Ranges can be expressed herein as from one particular value, and/or to another particular value. When such a range is expressed, also specifically contemplated and considered disclosed is the range from the one particular value and/or to the other particular value unless the context specifically indicates otherwise. It should be understood that all of the individual values and sub-ranges of values contained within an explicitly disclosed range are also specifically contemplated and should be considered disclosed unless the context specifically indicates otherwise. Further, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e., a single number) can be selected as the quantity, value, or feature to which the range refers.

As used herein, “AG transcription (Txn) Template” refers to a template that codes for “AG” at the +1 and +2 positions of the template, respectively.

As used herein, the term “complement,” “complementary,” or “complementarity” refers to specific base pairing between nucleotides or nucleic acids. Complementary nucleotides are, generally, A and T (or A and U), and G and C. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Complementarity, for example, between a capped oligonucleotide primer and a DNA template, may be “complete” or “total” where all of the nucleotide bases of two nucleic acid strands are matched according to recognized base pairing rules, it may be “partial” in which only some of the nucleotide bases of an initiating capped oligonucleotide primer and a DNA template are matched according to recognized base pairing rules, or it may be “absent” where none of the nucleotide bases of two nucleic acid strands are matched according to recognized base pairing rules. Complementarity can also be “substantial complementarity” where the nucleotide bases of two nucleic acids are matched according to recognized base pairing rules, but include one or more mismatches (e.g., 1, 2, 3, 4) from total complementarity.

As used herein, a “deoxyribonuclease (DNase)” is an enzyme that catalyzes the hydrolytic cleavage of phosphodiester linkages in the DNA backbone, thus degrading DNA. As used herein, the term “initiating capped oligonucleotide primer” refers to an oligonucleotide useful for initiating mRNA transcription. In certain embodiments, the initiating capped oligonucleotide primer may be a dinucleotide, a trinucleotide, or a tetranucleotide containing a 5′ to 5′ triphosphate linkage. In certain embodiments, the initiating capped oligonucleotide primer contains at least one nucleoside or phosphate modification. Certain initiating capped oligonucleotide primers include ^{m7}G_{3′Ome}ppp^{m6} A_{2′Ome}pG and ^{m7}Gppp^{m6} A_{2′Ome}pG.

“Inorganic pyrophosphatase” or “iPP” refers to an enzyme that catalyzes the conversion of one ion of pyrophosphate to two phosphate ions, thus inhibiting aggregation of pyrophosphate with magnesium ions during RNA polymerase (e.g., T7 RNA polymerase) transcription reactions.

As used herein, the term “in vitro” refers to a process that takes place outside a living organism (e.g., a multi-cellular organism, such as a human or a non-human animal), for example, in a test tube, culture dish, or elsewhere outside a living organism.

As used herein, the term “in vivo” refers to events that occur within a living organism.

As used herein, “messenger RNA transcript,” or “mRNA transcript,” is a transcript transcribed from a DNA template encoding a desired polypeptide. The mRNA transcript may contain coding and non-coding regions. For example, the DNA template can comprise an RNA polymerase promoter sequence, a 5′ UTR sequence, an open reading frame, and a 3′ UTR sequence. In some examples, the DNA template also comprises a nucleic acid sequence encoding a poly (A) tail.

“Nucleic acid” refers to nucleotides (e.g., deoxyribonucleotides or ribonucleotides) and polymers thereof in either single-, double- or multiple-stranded form, or complements thereof. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides include, for example, single and double stranded DNA, single and double stranded RNA, and hybrid molecules having mixtures of single and double stranded DNA and RNA. Examples of nucleic acid, e.g., polynucleotides include, but are not limited to, any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides, or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

As used herein, the term “nucleoside” refers to a nitrogenous base linked to a 5-carbon sugar (e.g., ribose or deoxyribose). The term includes all nucleosides, including all forms of nucleoside bases and furanosides. Base rings include purine and pyrimidine rings. Purine rings include, for example, adenine, guanine, and No-methyladenine. Pyrimidine rings include, for example, cytosine, thymine, 5-methylcytosine, and pseudouracil. Other nucleosides include, but are not limited to, ribo, 2′-O-methyl or 2′-deoxyribo derivatives of adenosine, guanosine, cytidine, thymidine, uridine, inosine, 7-methylguanosine or pseudouridine.

As used herein, the term “nucleoside triphosphate,” “nucleoside 5′ triphosphate” or “NTP” refers to a nucleoside linked to three phosphate groups. The term encompasses natural NTPs (for example, adenosine triphosphate (ATP), uridine triphosphate (UTP), guanine triphosphate (GTP), and cytosine triphosphate (CTP)) as well as modified NTPs.

As used herein, the term “modified NTP” refers to a nucleoside 5′-triphosphate having a chemical moiety group bound at any position, including the sugar, base, triphosphate chain, or any combination of these three locations. Optionally, the chemical moiety group may be any chemical moiety group compatible with the process of transcription. Examples of such NTPs include inosine triphosphate, dihyrouridine triphosphate, 2′-2′-fluoro-2′-deoxycytidine triphosphate, pseudouridine triphosphate, N1-methylpseudouridine triphosphate, and 5-methyluridine triphosphate, and can be found, for example, in “Nucleoside Triphosphates and Their Analogs: Chemistry, Biotechnology and Biological Applications,” Vaghefi, M., ed., Taylor and Francis, Boca Raton (2005).

As used herein, the term “modified oligonucleotide” or “modified trinucleotide” includes, for example, an oligonucleotide containing a modified nucleoside, a modified internucleotide linkage, or having any combination of modified nucleosides and internucleotide linkages. Examples of internucleotide linkage modifications include phosphorothioate, phosphotriester and methylphosphonate derivatives (Stec, W. J., et al., Chem. Int. Ed. Engl., 33:709-722 (1994); Lebedev, A. V., et al., E., Perspect. Drug Discov. Des., 4:17-40 (1996); and Zon, et al., U.S. patent application No. 20070281308). Other examples of internucleotide linkage modifications may be found in Waldner, et al., Bioorg. Med. Chem. Letters 6:2363-2366 (1996).

As used herein, “oligo dT purification” is an affinity chromatography method for purification of mRNA that terminates in a poly-A tail.

A “primary RNA” or “primary RNA transcript” means the RNA molecule that is newly synthesized by an RNA polymerase in vitro and which RNA molecule has a triphosphate on the 5′-carbon of its most 5′ nucleotide.

As used herein, the term “prematurely aborted RNA transcript” refers to incomplete products of an in vitro transcription reaction. Prematurely aborted RNA sequences may be any length that is less than the intended length of the desired transcriptional product.

The term “promoter” as used herein refers to a nucleotide sequence in a DNA template that directs and controls the initiation of transcription of a particular DNA sequence. Promoters are typically immediately adjacent to (or partially overlap with) the DNA sequence to be transcribed. Promoter sequences are typically located directly upstream or at the 5′ end of the transcription initiation site.

As used herein, the term “purified” or “purify” refers to separating a substance from at least some of the components (e.g., impurities or contaminants) with which it was associated when initially produced. For example, RNA transcripts are purified by removal of contaminating proteins or other undesired nucleic acid species (e.g., double-stranded RNA, DNA, and/or incomplete or aborted RNA transcripts). Purified substances (e.g., capped mRNA transcripts) can be separated from 10%, 20%, 30%, 40%, 50%, 60%, 70%, 80%, 90%, 91%, 92%, 93%, 94%, 95%, 96%, 97%, 98%, 99%, or more than 99% of the other components with which they were initially associated.

As used herein, the term “RNase inhibitor” or “ribonuclease inhibitor” refers to a molecule that inhibits RNase activity, for example, during an in vitro transcription reaction.

As used herein, the term “RNA polymerase” refers to an enzyme that synthesizes RNA from a DNA template. For in vitro transcription methods, single subunit phage RNA polymerases derived from T7, T3, SP6, K1-5, KIE, KIF or K11 bacteriophages, or variants thereof, are typically used. This family of polymerases has simple, minimal promoter sequences of about 17 nucleotides which require no accessory proteins and have minimal constraints on the initiating nucleotide sequence.

Salts of one or more compounds as described herein (e.g., primers) can be used in the disclosed methods. The term “salt(s),” as used herein, refers to derivatives of the compounds described herein prepared by the reaction of an acidic or basic moiety of the compound with a mineral or organic acid or base. Optionally, the salts can be pharmaceutically acceptable salts. As used herein, the term “pharmaceutically acceptable salt(s)” refers to those salts of the compounds described herein or derivatives thereof that are, within the scope of sound medical judgment, suitable for use in contact with the tissues of subjects without undue toxicity, irritation, allergic response, and the like, commensurate with a reasonable benefit/risk ratio, and effective for their intended use, as well as the zwitterionic forms, where possible, of the compounds described herein. These salts can be prepared in situ during the isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate, and laurylsulphonate salts, and the like. Salts may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See S. M. Barge et al., J. Pharm. Sci. (1977) 66, 1; and Remington: The Science and Practice of Pharmacy, 23d Edition, Adejare et al. eds., Academic Press (2020); which are incorporated herein by reference in their entireties.)

As used herein, the term “substantially free” refers to a state in which relatively little or no amount of an undesired substance (e.g., prematurely aborted RNA sequences, DNA, and/or double-stranded RNA) is present in a sample. “Substantially free of impurities” means impurities are present at a level less than approximately 5%, 4%, 3%, 2%, 1.0%, 0.9%, 0.8%, 0.7%, 0.6%, 0.5%, 0.4%, 0.3%, 0.2%, 0.1% or less (w/w) in a sample. For example, “substantially free of double-stranded RNA” means double-stranded RNA is present at a level less than approximately 5%, 4%, 3%, 2%, 1.0%, 0.9%, 0.8%, 0.7%, 0.6%, 0.5%, 0.4%, 0.3%, 0.2%, 0.1% or less (w/w) in a sample.

As used herein, “tangential flow filtration (TFF)” is a type of filtration wherein the material to be purified is passed tangentially across a filter rather than through it. In TFF, undesired permeate passes through the filter, while the desired retentate passes along the filter and is collected downstream. In TFF, the desired material is typically contained in the retentate, which is the opposite of what is encountered when performing traditional membrane or dead-end filtration.

As used herein, the term “transcription” refers to enzymatically making or synthesizing RNA that is complementary to a DNA template, thereby producing a number of RNA complements of a DNA sequence. The RNA molecule synthesized in a transcription reaction is an “RNA transcript,” “primary transcript,” or “transcript.” Transcription reactions involving the compositions and methods provided herein employ initiating capped oligonucleotide primers described herein. Certain initiating capped oligonucleotide primers include ^{m7}G_{3′Ome}ppp^{m6} A_{2′Ome}pG and ^{m7}Gppp^{m6} A_{2′Ome}pG. Transcription of a DNA template may be exponential, nonlinear or linear. A DNA template may be a double-stranded linear DNA, a partially double-stranded linear DNA, circular double-stranded DNA, DNA plasmid, PCR amplified product, or a modified nucleic acid template that is compatible with RNA polymerase.

As used herein, “uridine depleted (UD)” refers to an mRNA transcript that has been modified to reduce the number of uridines in the mRNA transcript, for example, by using synonymous codons, i.e., codons that encode the same amino acid. Some uridine depleted mRNA transcripts can also be chemically modified, for example, by insertion of modified triphosphates (e.g., pseudouridine or 5-methoxyuridine) during in vitro transcription.

As used herein, “capped mRNA” refers to an mRNA transcript that contains an N7-methyl guanosine connected to the 5′ nucleotide through a 5′ to 5′ triphosphate linkage. This may occur when an initiating capped oligonucleotide primer initiates the 5′ start of a transcript formation by polymerase. Furthermore, the 5′ cap of an mRNA, referred to as “Cap1,” means an N7-methyl guanosine connected to the 5′ nucleotide through a 5′ to 5′ triphosphate linkage and a methylation on the 2′ position of the first nucleotide of the mRNA sequence.

As used herein, “uncapped” refers to an mRNA transcript that does not contain an N7-methyl guanosine connected to the 5′ nucleotide through a 5′ to 5′ triphosphate linkage. This may occur when an NTP initiates the 5′ start of the transcript resulting in a 5′ triphosphate. In certain methods this 5′ end is phosphatase-treated for analysis of a 5′ OH.

As used herein, the term “cap analog” means a structural derivative of an RNA cap.

As used herein, “capping efficiency” refers to the percentage of mRNA molecules produced via in vitro transcription which contain a Cap1 structure as compared to mRNA molecules produced via in vitro transcription that do not contain a Cap1 structure. The formula for calculating percent capping efficiency is: Σ mass intensities of identified 5′ capped species/(Σ mass intensities of identified 5′ capped+5′ uncapped species)×100.

As used herein, “Spike-in” or “Spike” mix refers to an addition of reaction buffer (i.e., Transcription Buffer used in the first step of the transcription process) and NTPs halfway through total reaction time of the transcription process. In embodiments, the reaction buffer is Comparative Buffer. In embodiments, the reaction buffer is Buffer 1. In embodiments, the “Spike-in” takes place after two hours of incubating the reaction mixture used for the transcription process, and “Spike-in” mix is allowed to incubate for two additional hours. The “Spike-in” improves yield and capping efficiency of the transcribed mRNA and reduces relative dsRNA levels.

As used herein, “Final Reaction Mixture” refers to the solution comprising capped mRNA transcripts after addition of “Spike-in”.

As used herein, “Fixed” refers to a standard transcription process, which does not include a “Spike-in”, with incubation of the reaction mixture for 2-3 hours.

As used herein, “Transcription Buffer” also referred to as “Buffer 1 IVT” or “Buffer 1” refers to a new transcription buffer developed herein for optimal yield and capping efficiency of the mRNA transcripts and minimal dsRNA levels. “10× Transcription Buffer” refers to 10× concentrated Transcription Buffer, which is then diluted 1:10 in the reaction mixture to 1× Transcription Buffer during transcription process. The components of “Transcription Buffer” also referred to as “Buffer 1 IVT” are provided in the specification and the Examples section.

In Vitro mRNA Synthesis

Provided herein are in vitro methods for synthesizing high yields of capped messenger RNA (mRNA) transcripts (e.g., 7-13 mg/ml) that are substantially free of impurities such as DNA, protein, double-stranded RNA and/or incomplete mRNA transcripts. In embodiments, less than 1% of the mass of the synthesized mRNA transcripts is double-stranded RNA. In embodiments, less than 0.0005% of the nucleic acid mass in a sample of synthesized mRNA transcripts is DNA.

In an aspect, provided herein is an in vitro method for synthesizing capped messenger RNA (mRNA) transcripts comprising (a) forming a reaction mixture comprising: (i) 6.0 mM to 25 mM initiating capped oligonucleotide primer or a salt thereof; (ii) 0.020 μg/μl to 0.1 μg/μl DNA template; (iii) nucleoside triphosphates (NTPs), including 3 mM to 7 mM ATP; 3 mM to 7 mM CTP; 3 mM to 7 mM GTP; and 3 mM to 7 mM UTP; (iv) 1× Transcription Buffer; (v) 10 U/μL to 18 U/μL RNA polymerase; and (vi) DNase and RNase free water, wherein the reaction mixture has a pH of 6.0 to 8.0; and (b) incubating the reaction mixture, for 2-3 hours, under conditions that allow transcription of the DNA template to obtain a solution comprising capped mRNA transcripts.

In embodiments, the method further comprises an RNase inhibitor and/or an inorganic pyrophosphatase.

In embodiments, the method further comprises a Spike-in mix, which includes incubating the solution comprising capped mRNA transcripts with: (i) nucleoside triphosphates (NTPs), including ATP, CTP, GTP, and UTP, each with a concentration of 3 mM to 8 mM in a Final Reaction Mixture; and (ii) 10× Transcription Buffer, with a concentration of 1× Transcription Buffer in a Final Reaction Mixture. In embodiments, one or more of the NTPs is a modified NTP.

In embodiments, the method further comprises a Spike-in mix, which includes incubating the solution comprising capped mRNA transcripts with: (i) nucleoside triphosphates (NTPs), including ATP, CTP, GTP, and UTP, each with a concentration of 3 mM to 8 mM in the Final Reaction Mixture; and (ii) 10× Transcription Buffer, with a concentration of 1× Transcription Buffer in the Final Reaction Mixture, wherein the incubation is performed at 37° C. for 1 to 3 hours.

In embodiments, the method further comprises incubating the solution comprising capped mRNA transcripts or the Final Reaction Mixture with: (i) a solution of CaCl₂) with a concentration of 0.5 mM to 5 mM in the reaction mixture or the Final Reaction Mixture; and (ii) DNase I.

In embodiments, the method further comprises incubating the solution comprising capped mRNA transcripts with: (i) a solution of CaCl₂) with a concentration of 0.5 mM to 5 mM in the reaction mixture; and (ii) DNase I.

In embodiments, the method further comprises incubating the Final Reaction Mixture with: (i) a solution of CaCl₂) with a concentration of 0.5 mM to 5 mM in the Final Reaction Mixture; and (ii) DNase I.

In embodiments, the method further comprises subjecting the DNase treated reaction mixture to eliminate proteins from the in vitro transcription reaction, for example, by using Proteinase K treatment. Exemplary conditions for Proteinase K treatment include treating a sample with 0.1 mg/mL proteinase K in the presence of 0.5% SDS and 5 mM DTT, for 60 min+/−10 min at 37° C.

In embodiments, the method further comprises subjecting the DNase treated reaction mixture to phosphatase treatment. Exemplary conditions for phosphatase treatment include formulating an mRNA sample in 1× Antarctic phosphatase reaction buffer, 0.5 U/μL RNase inhibitor, and 0.25 units of Antarctic phosphatase per μg of mRNA, then incubating at 37° C. for 60 min+/−10 min.

In embodiments, the method further comprises subjecting the DNase treated reaction mixture to one or more purification steps. The mRNA transcripts produced by the methods described herein can be purified using one or more purification techniques known to those of skill in the art. See, Baronti et al. “A guide to large-scale RNA sample preparation,” Anal. Bioanal. Chem. 410 (14): 3239-33252 (2018). For example, the mRNAs can be purified by liquid chromatography (e.g., reversed-phase ion pairing HPLC (RP-IP-HPLC), anion-exchange chromatography, cation exchange chromatography, affinity chromatography, size-exclusion chromatography), precipitation, RNeasy purification, diafiltration, tangential flow filtration, oligo dT chromatography, silica membrane purification, or hydrophobic interaction chromatography, to name a few.

In embodiments, the one or more purification steps comprise oligo dT purification. See Green and Sambrrok “Isolation of Poly (A)⁺ Messenger RNA Using Magnetic Oligo (dT) Beads,” Cold Spring Harb. Protocol. 2019 Oct. 1; 2019 (10). doi: 10.1101/pdb.prot101733; and Sau et al. “Automated solid-phase synthesis of high capacity oligo-dT cellulose for affinity purification of poly-A tagged biomolecules,” Bioorg Med Chem Lett. 2014 Dec. 15; 24 (24): 5692-5694.

In embodiments, the one or more purification steps comprise tangential flow filtration. Exemplary parameters and devices used for TFF are described in Michaels et al., “Tangential Flow Filtration” in Separations Technology, Pharmaceutical and Biotechnology Applications (W. P. Olson, ed., Interpharm Press, Inc., Buffalo Grove, Ill. 1995). Transmembrane pressure, feed rate, and flow rate of the permeate are important variables in a typical TFF process. The transmembrane pressure is the force that drives fluid through the filter, carrying with it permeable molecules. Typically, transmembrane pressure ranges between 1 and 30 pounds per square inch. In embodiments, the transmembrane pressure is less than 20 psi, less than 10 psi, or less than 5 psi.

The feed rate (also known as the crossflow velocity) is the rate of the solution flow through the feed channel and across the filter. The feed rate determines the force that sweeps away molecules that may restrict filtrate flow. In embodiments, the feed rate is between 50 and 2500 mL/min. In some methods, the feed rate is between 100 and 2500 mL/min, 200 and 2500 mL min, 300 and 2500 mL/min, 400 and 2500 mL/min, 500 and 2500 mL/min, 600 and 2500 mL min, 700 and 2500 mL/min, 800 and 2500 mL/min, 900 and 2500 mL/min, or 1000 and 2500 mL/min. In some methods, the feed rate is 500 mL/min or greater, 600 mL/min or greater, 700 mL/min or greater, 800 mL/min or greater, 900 mL/min or greater, 1000 mL/min or greater, 1500 mL/min or greater, or 2000 mL/min or greater.

The flow rate of the permeate is the rate at which the permeate is removed from the system. In some methods, the flow rate is selected from 100 to 800 mL/min, 100 to 700 mL/minute, 100 to 600 mL/minute, 100 to 500 mL/min, 100 to 400 mL/min, and 100 to 300 mL/min. In some methods, the permeate flow rate is greater than 100 mL/min, 200 mL/min, 300 mL/min, 400 mL/min, 500 mL/min, 600 mL/min or 700 mL/min. Any combination of pressure, feed rate and flow rate can be used in any of the TFF purification methods described herein.

In embodiments, the one or more purification steps yield purified, capped mRNA transcripts that are substantially free of impurities.

In embodiments, the DNase treated reaction mixture is subjected to oligo dT purification to obtain oligo dT purified mRNA transcripts. In such embodiments, it is not necessary to subject the DNase treated reaction mixture to a tangential flow filtration step prior to the oligo dT purification step. Some embodiments further comprise subjecting the oligo dT purified mRNA transcripts to tangential flow filtration to obtain purified, capped mRNA transcripts. In some embodiments, the one or more purification steps comprise a first tangential flow filtration step, followed by an oligo dT purification step, and a second tangential flow filtration step.

In an aspect, provided herein is an in vitro method for synthesizing capped messenger RNA (mRNA) transcripts comprising: (a) forming a reaction mixture comprising: (i) 6.0 mM to 25 mM initiating capped oligonucleotide primer or a salt thereof; (ii) 0.020 μg/μl to 0.1 μg/μl DNA template; (iii) nucleoside triphosphates (NTPs), including 3 mM to 7 mM ATP; 3 mM to 7 mM CTP; 3 mM to 7 mM GTP; and 3 mM to 7 mM UTP; (iv) 1× Transcription Buffer; (v) 10 U/μL to 18 U/μL RNA polymerase; and (vi) DNase and RNase free water, wherein the reaction mixture has a pH of 6.0 to 8.0; (b) incubating the reaction mixture, for 2-3 hours, under conditions that allow transcription of the DNA template to obtain a solution comprising capped mRNA transcripts; and (c) incubating the solution comprising capped mRNA transcripts with: (i) a solution of CaCl₂) with a concentration of 0.5 mM to 5 mM in the reaction mixture; and (ii) DNase I, wherein the incubation is performed at 37° C. for 10 to 60 minutes.

In an aspect, provided herein is an in vitro method for synthesizing capped messenger RNA (mRNA) transcripts comprising: (a) forming a reaction mixture comprising: (i) 6.0 mM to 25 mM initiating capped oligonucleotide primer or a salt thereof; (ii) 0.020 μg/μl to 0.1 μg/μl DNA template; (iii) nucleoside triphosphates (NTPs), including 3 mM to 7 mM ATP; 3 mM to 7 mM CTP; 3 mM to 7 mM GTP; and 3 mM to 7 mM UTP; (iv) 1× Transcription Buffer; (v) 10 U/μL to 18 U/μL RNA polymerase; and (vi) DNase and RNase free water, wherein the reaction mixture has a pH of 6.0 to 8.0; (b) incubating the reaction mixture, for 2-3 hours, under conditions that allow transcription of the DNA template to obtain a solution comprising capped mRNA transcripts; (c) optionally incubating the solution comprising capped mRNA transcripts with a Spike-in mix: (i) nucleoside triphosphates (NTPs), including ATP, CTP, GTP, and UTP, each with a concentration of 3 mM to 8 mM in the Final Reaction Mixture; and (ii) 10× Transcription Buffer, with a concentration of 1× Transcription Buffer in the Final Reaction Mixture, wherein the incubation is performed at 37° C. for 1 to 3 hours; and (d) incubating the Final Reaction Mixture with: (i) a solution of CaCl₂) with a concentration of 0.5 mM to 5 mM in the Final Reaction Mixture; and (ii) DNase I, wherein the incubation is performed at 37° C. for 10 to 60 minutes.

In an aspect, provided herein is an in vitro method for synthesizing capped messenger RNA (mRNA) transcripts comprising: (a) forming a reaction mixture comprising: (i) 6.0 mM to 25 mM initiating capped oligonucleotide primer or a salt thereof; (ii) 0.020 μg/μl to 0.1 μg/μl DNA template; (iii) nucleoside triphosphates (NTPs), including 3 mM to 7 mM ATP; 3 mM to 7 mM CTP; 3 mM to 7 mM GTP; and 3 mM to 7 mM UTP; (iv) 1× Transcription Buffer; (v) 10 U/μL to 18 U/μL RNA polymerase; and (vi) DNase and RNase free water, wherein the reaction mixture has a pH of 6.0 to 8.0; (b) incubating the reaction mixture, for 2-3 hours, under conditions that allow transcription of the DNA template to obtain a solution comprising capped mRNA transcripts; (c) incubating the solution comprising capped mRNA transcripts with a Spike-in mix: (i) nucleoside triphosphates (NTPs), including ATP, CTP, GTP, and UTP, each with a concentration of 3 mM to 8 mM in the Final Reaction Mixture; and (ii) 10× Transcription Buffer, with a concentration of 1× Transcription Buffer in the Final Reaction Mixture, wherein the incubation is performed at 37° C. for 1 to 3 hours; and (d) incubating the Final Reaction Mixture with: (i) a solution of CaCl₂) with a concentration of 0.5 mM to 5 mM in the Final Reaction Mixture; and (ii) DNase I, wherein the incubation is performed at 37° C. for 10 to 60 minutes.

In embodiments, the method further comprises subjecting the DNase-treated reaction mixture to one or more purification step. In embodiments, the method further comprises subjecting the DNase-treated reaction mixture to tangential flow filtration. In embodiments, the method further comprises subjecting the DNase-treated reaction mixture to oligo dT purification. In embodiments, the method further comprises subjecting the DNase-treated reaction mixture to lithium chloride (LiCl) precipitation. In embodiments, the method further comprises subjecting the DNase-treated reaction mixture to RNeasy purification.

In embodiments, the method further comprises subjecting the DNase-treated reaction mixture to two purification steps. In embodiments, the method further comprises subjecting the DNase-treated reaction mixture to three purification steps. In embodiments, the method further comprises subjecting the DNase-treated reaction mixture to four purification steps.

In embodiments, the two purification steps are the same. In embodiments, the two purification steps are different. In embodiments, the three purification steps are the same. In embodiments, the three purification steps are different. In embodiments, two purification steps are the same and one is different.

In embodiments, the method further comprises (e) subjecting the DNase-treated reaction mixture to tangential flow filtration to obtain a first sample comprising purified, capped mRNA transcripts; (f) subjecting the first sample from step (e) to oligo dT purification to obtain a second sample comprising purified, capped mRNA transcripts; and (g) subjecting the second sample from step (f) to a second tangential flow filtration step to obtain a third sample comprising purified, capped mRNA transcripts. In embodiments, the first, second, and/or third sample comprising purified, capped mRNA transcripts are substantially free of impurities.

As set forth above, the methods described herein can be used to efficiently synthesize capped mRNAs. In embodiments, greater than 95%, 96%, 97%, 98%, or 99% of the synthesized mRNA transcripts are capped mRNA transcripts. In embodiments, greater than 95% of the synthesized mRNA transcripts are capped mRNA transcripts. In embodiments, greater than 96% of the synthesized mRNA transcripts are capped mRNA transcripts. In embodiments, greater than 97% of the synthesized mRNA transcripts are capped mRNA transcripts. In embodiments, greater than 98% of the synthesized mRNA transcripts are capped mRNA transcripts. In embodiments, greater than 99% of the synthesized mRNA transcripts are capped mRNA transcripts.

It is understood that the mRNA transcripts produced by the methods described herein can be of any length. In embodiments, the synthesized, capped mRNA transcripts are between 0.3 kb to 10 kb in length. For example, the mRNA transcript can have a length selected from can be selected from 0.3 kb to 10 kb, 0.4 kb to 10 kb, 0.5 kb to 10 kb, 0.6 kb to 10 kb, 0.7 kb to 10 kb, 0.8 kb to 10 kb, 0.9 kb to 10 kb, 1 kb to 10 kb, 1.1 kb to 10 kb, 1.2 kb to 10 kb, 1.3 kb to 10 kb, 1.4 kb to 10 kb, 1.5 kb to 10 kb, 1.6 kb to 10 kb, 1.7 kb to 10 kb, 1.8 kb to 10 kb, 1.9 kb to 10 kb, 2 kb to 10 kb, 2.1 kb to 10 kb, 2.2 kb to 10 kb, 2.3 kb to 10 kb, 2.4 kb to 10 kb, 2.5 kb to 10 kb, 2.6 kb to 10 kb, 2.7 kb to 10 kb, 2.8 kb to 10 kb, 2.9 kb to 10 kb, 3 kb to 10 kb, 3.1 kb to 10 kb, 3.2 kb to 10 kb, 3.3 kb to 10 kb, 3.4 kb to 10 kb, 3.5 kb to 10 kb, 3.6 kb to 10 kb, 3.7 kb to 10 kb, 3.8 kb to 10 kb, 3.9 kb to 10 kb, 4 kb to 10 kb, 4.1 kb to 10 kb, 4.2 kb to 10 kb, 4.3 kb to 10 kb, 4.4 kb to 10 kb, 4.5 kb to 10 kb, 4.6 kb to 10 kb, 4.7 kb to 10 kb, 4.8 kb to 10 kb, 4.9 kb to 10 kb, 5 kb to 10 kb, 5.1 kb to 10 kb, 5.2 kb to 10 kb, 5.3 kb to 10 kb, 5.4 kb to 10 kb, 5.5 kb to 10 kb, 5.6 kb to 10 kb, 5.7 kb to 10 kb, 5.8 kb to 10 kb, 5.9 kb to 10 kb, 6 kb to 10 kb, 6.1 kb to 10 kb, 6.2 kb to 10 kb, 6.3 kb to 10 kb, 6.4 kb to 10 kb, 6.5 kb to 10 kb, 6.6 kb to 10 kb, 6.7 kb to 10 kb, 6.8 kb to 10 kb, 6.9 kb to 10 kb, 7 kb to 10 kb, 7.1 kb to 10 kb, 7.2 kb to 10 kb, 7.3 kb to 10 kb, 7.4 kb to 10 kb, 7.5 kb to 10 kb, 7.6 kb to 10 kb, 7.7 kb to 10 kb, 7.8 kb to 10 kb, 7.9 kb to 10 kb, 8.0 kb to 10 kb, 8.1 kb to 10 kb, 8.2 kb to 10 kb, 8.3 kb to 10 kb, 8.4 kb to 10 kb, 8.5 kb to 10 kb, 8.6 kb to 10 kb, 8.7 kb to 10 kb, 8.8 kb to 10 kb, 8.9 kb to 10 kb, 9.0 kb to 10 kb, 9.1 kb to 10 kb, 9.2 kb to 10 kb, 9.3 kb to 10 kb, 9.4 kb to 10 kb, 9.5 kb to 10 kb, 9.6 kb to 10 kb, 9.7 kb to 10 kb, 9.8 kb to 10 kb, 9.9 kb to 10 kb.

In embodiments, the mRNA transcript is 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10 kb in length.

It is understood that the in vitro transcription methods described herein can be performed at any scale. For example, the reaction volume for in vitro mRNA synthesis can be from 0.1 ml to 10 liters (L). In some cases, the reaction volume is selected from 0.1 ml to 5 ml, 5 ml to 10 ml, 15 ml to 20 ml, 20 ml to 50 ml, 50 ml to 100 ml, 100 ml to 200 ml, 200 ml to 300 ml, 300 ml to 400 ml, 500 ml to 600 ml, 600 ml to 700 ml, 700 ml to 800 ml, 800 ml to 900 ml, 900 ml to 1 L, 20 ml to 5 L, 50 ml to 5 L, 100 ml to 5 L, 200 ml to 5 L, 300 ml to 5 L, 400 ml to 5 L, 500 ml to 5 L, 1 L to 5 L, 1.5 L to 5 L, 2 L to 5 L, 2.5 L to 5 L, 3.0 L to 5 L, 3.5 L to 5 L, 4.0 to 5 L, 4.5 to 5 L, 1 L to 2 L, 2 L to 3 L, 3 L to 4 L, 4 L to 5 L, 5 L to 6 L, 6 L to 7 L, 7 L to 8 L, 8 L to 9 L, and 9 L to 10 L.

In embodiments, the initiating capped oligonucleotide primer is a dinucleotide. In embodiments, the initiating capped oligonucleotide primer is a trinucleotide. In embodiments, the initiating capped oligonucleotide primer is a tetranucleotide.

In embodiments, the initiating capped oligonucleotide primer comprises a 5′ to 5′ triphosphate bridge. In embodiments, the initiating capped oligonucleotide primer comprises a 5′ to S′ diphosphate bridge. In embodiments, the initiating capped oligonucleotide primer comprises a S′ to 5′ monophosphate bridge. In embodiments, the initiating capped oligonucleotide primer comprises a S′ to S′ modified bridge. In embodiments, the bridge can be phosphorothioate, phosphotriester or methylphosphonate derivative.

In embodiments, the initiating capped oligonucleotide primer is ^{m7}Gppp^{m6} A_{2′Ome}pG, which in its anionic form, has the following structure:

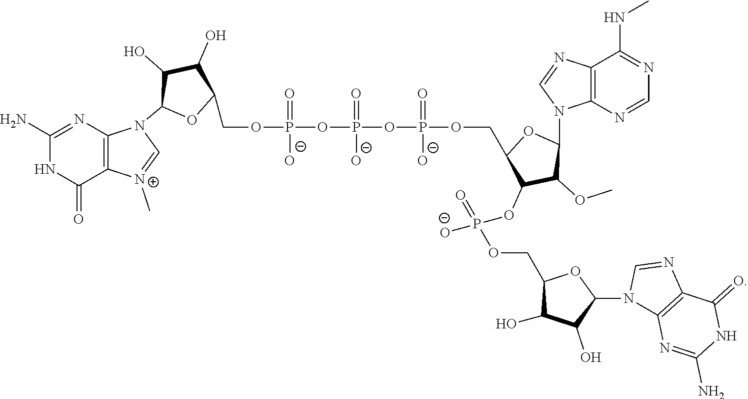

In embodiments, the initiating capped oligonucleotide primer is ^{m7}G_{3′Ome}ppp^{m6} A_{2′Ome}pG, which in its anionic form, has the following structure:

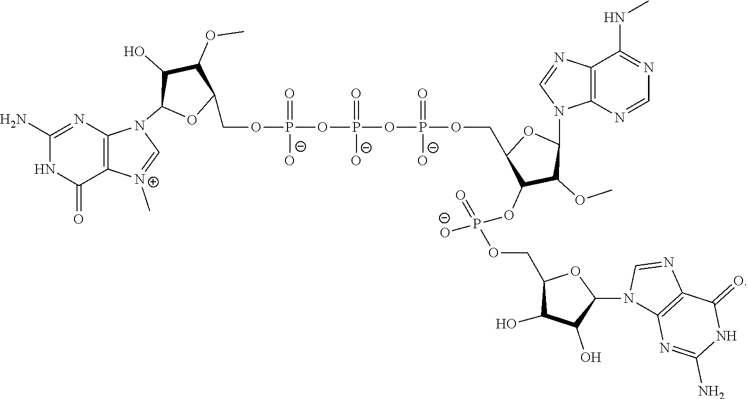

^{m7}Gppp^{m6} A_{2′Ome}pG and ^{m7}G_{3′Ome}ppp^{m6} A_{2′Ome}pG are initiating capped primers that can be extended by RNA polymerase through the incorporation of an NTP onto the 3′-end of the primer. ^{m7}Gppp^{m6} A_{2′Ome}pG and ^{m7}G_{3′Ome}ppp^{m6} A_{2′Ome}pG are substantially complementary to the template DNA sequence at the transcription initiation site. The initiating capped oligonucleotide initiates in vitro transcription under the control of a promoter in a transcription system containing a DNA template (e.g., linearized plasmid or PCR product), RNA polymerase, nucleoside 5′-triphosphates and appropriate buffer.

In the methods provided herein, once the capped primer is incorporated into an in vitro transcribed mRNA, the in vitro transcribed RNA can comprise a 5′ cap comprising ^{m7}Gppp^{m16} A_{2′Ome}pG or ^{m7}G_{3′Ome}ppp^{m6} A_{2′Ome}pG, a 5′ untranslated region (5′ UTR), a sequence encoding a polypeptide of interest, a 3′ untranslated region (3′ UTR), and optionally a poly (A) tail. One of skill in the art would understand that a poly (A) tail can be encoded by the DNA template such that the synthesized mRNA comprises a poly(A) tail, or be attached to an RNA after in vitro transcription.

In embodiments, an mRNA comprising a ^{m7}Gppp^{m6} A_{2′Ome}pG cap structure is represented according to the following chemical structure:

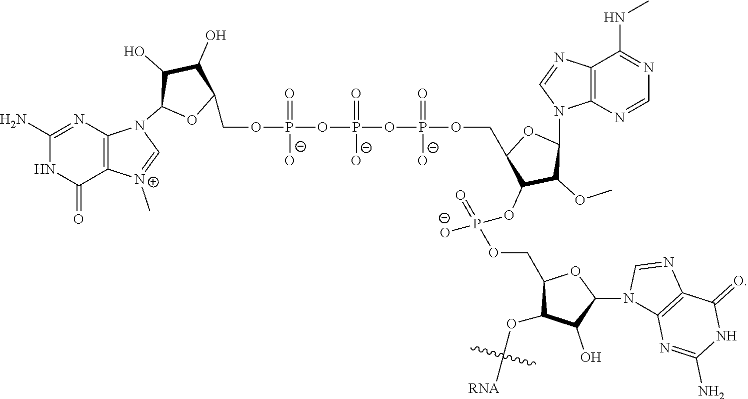

In some examples, the mRNA comprising a ^{m7}G_{3′Ome}ppp^{m6} A_{2′Ome}pG cap structure is represented according to the following chemical structure:

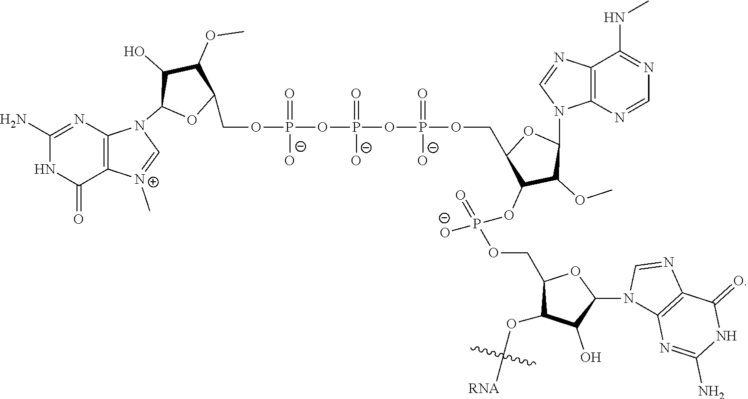

As understood by those of skill in the art, the structures shown of the compounds described herein are representations of one form of the compound. Although such compounds may be drawn or described in protonated (free acid) form, in ionized (anion) form, or ionized and in association with a cation (salt) form, aqueous solutions of such compounds exist in equilibrium among such forms. For example, a phosphate linkage of a compound described herein, in aqueous solution, exists in equilibrium among free acid, anion, and salt forms. Unless otherwise indicated, compounds described herein are intended to include all such forms. Moreover, certain compounds have several such linkages, each of which is in equilibrium. Thus, compounds in solution exist in an ensemble of forms at multiple positions all at equilibrium. Drawn structures necessarily depict a single form. Nevertheless, unless otherwise indicated, such drawings are likewise intended to include all corresponding forms. Herein, a structure depicting the free acid of a compound followed by the term “or salts thereof” expressly includes all such forms that may be fully or partially protonated/de-protonated/in association with a cation. In certain instances, one or more specific cation(s) is identified. In embodiments, compounds disclosed herein are in aqueous solution with sodium. In embodiments, compounds are in aqueous solution with potassium. In embodiments, compounds are in aqueous solution with lithium. In embodiments, compounds are in aqueous solution with triethylammonium. In embodiments, compounds are in PBS. In embodiments, compounds are in water. In such embodiments, the pH of the solution is adjusted with NaOH and/or HCl to achieve a desired pH.

In embodiments, the reaction mixture comprises 6.0 mM to 25 mM initiating capped oligonucleotide primer. For example, in embodiments, the reaction mixture comprises initiating capped oligonucleotide primer in a concentration selected from 6.0 mM to 25.0 mM, 6.5 mM to 25.0 mM, 7.0 mM to 25.0 mM, 7.5 mM to 25.0 mM, 8.0 mM to 25.0 mM, 8.5 mM to 25.0 mM, 9.0 mM to 25.0 mM, 9.5 mM to 25.0 mM, 10.0 mM to 25.0 mM, 10.5 mM to 25.0 mM, 11.0 mM to 25.0 mM, 11.5 mM to 25.0 mM, 12.0 mM to 25.0 mM, 12.5 mM to 25.0 mM, 13.0 mM to 25.0 mM, 13.5 mM to 25.0 mM, 14.0 mM to 25.0 mM, 14.5 mM to 25.0 mM, 15.0 mM to 25.0 mM, 15.5 mM to 25.0 mM, 16.0 mM to 25.0 mM, 16.5 mM to 25.0 mM, 17.0 mM to 25.0 mM, 17.5 mM to 25.0 mM, 18.0 mM to 25.0 mM, 18.5 mM to 25.0 mM, 19.0 mM to 25.0 mM, 19.5 mM to 25.0 mM, 20.0 mM to 25.0 mM, 20.5 mM to 25.0 mM, 21.0 mM to 25.0 mM, 21.5 mM to 25.0 mM, 22.0 mM to 25.0 mM, 22.5 mM to 25.0 mM, 23.0 mM to 25.0 mM, 23.5 mM to 25.0 mM, 24.0 mM to 25.0 mM, 24.5 mM to 25.0 mM, 6.0 mM to 20.0 mM, 6.5 mM to 20.0 mM, 7.0 mM to 20.0 mM, 7.5 mM to 20.0 mM, 8.0 mM to 20.0 mM, 8.5 mM to 20.0 mM, 9.0 mM to 20.0 mM, 9.5 mM to 20.0 mM, 10.0 mM to 20.0 mM, 10.5 mM to 20.0 mM, 11.0 mM to 20.0 mM, 11.5 mM to 20.0 mM, 12.0 mM to 20.0 mM, 12.5 mM to 20.0 mM, 13.0 mM to 20.0 mM, 13.5 mM to 20.0 mM, 14.0 mM to 20.0 mM, 14.5 mM to 20.0 mM, 15.0 mM to 20.0 mM, 15.5 mM to 20.0 mM, 16.0 mM to 20.0 mM, 16.5 mM to 20.0 mM, 17.0 mM to 20.0 mM, 17.5 mM to 20.0 mM, 18.0 mM to 20.0 mM, 18.5 mM to 20.0 mM, 19.0 mM to 20.0 mM, 19.5 mM to 20.0 mM, 6.0 mM to 15.0 mM, 6.5 mM to 15.0 mM, 7.0 mM to 15.0 mM, 7.5 mM to 15.0 mM, 8.0 mM to 15.0 mM, 8.5 mM to 15.0 mM, 9.0 mM to 15.0 mM, 9.5 mM to 15.0 mM, 10.0 mM to 15.0 mM, 10.5 mM to 15.0 mM, 11.0 mM to 15.0 mM, 11.5 mM to 15.0 mM, 12.0 mM to 15.0 mM, 12.5 mM to 15.0 mM, 13.0 mM to 15.0 mM, 13.5 mM to 15.0 mM, 14.0 mM to 15.0 mM, and 14.5 mM to 15.0 mM initiating capped primer. In embodiments, the reaction mixture comprises 6.0 mM, 6.5 mM, 7.0 mM, 7.5 mM, 8.0 mM, 8.5 mM, 9.0 mM, 9.5 mM, 10.0 mM, 10.5 mM, 11.0 mM, 11.5 mM, 12.0 mM, 12.5 mM, 13.0 mM, 13.5 mM, 14.0 mM, 14.5 mM, 15.0 mM, 15.5 mM, 16.0 mM, 16.5 mM, 17.0 mM, 17.5 mM, 18.0 mM, 18.5 mM, 19.0 mM, 19.5 mM, 20.0 mM, 20.5 mM, 21.0 mM, 21.5 mM, 22.0 mM, 22.5 mM, 23.0 mM, 23.5 mM, 24.0 mM, 24.5 mM, or 25.0 mM initiating capped primer.

In embodiments, the reaction mixture comprises 0.020 μg/μl to 0.1 μg/μl DNA template. In embodiments, the concentration of the DNA template may be selected from 0.020 μg/μl to 0.1 μg/μl, 0.030 μg/μl to 0.1 μg/μl, 0.040 μg/μl to 0.1 μg/μl, 0.050 μg/μl to 0.1 μg/μl, 0.060 μg/μl to 0.1 μg/μl, 0.070 μg/μl to 0.1 μg/μl, 0.080 μg/μl to 0.1 μg/μl, 0.090 μg/μl to 0.1 μg/μl, 0.020 μg/μl to 0.09 μg/μl, 0.030 μg/μl to 0.09 μg/μl, 0.040 μg/μl to 0.09 μg/μl, 0.050 μg/μl to 0.09 μg/μl, 0.060 μg/μl to 0.09 μg/μl, 0.070 μg/μl to 0.09 μg/μl, 0.080 μg/μl to 0.09 μg/μl, 0.020 μg/μl to 0.08 μg/μl, 0.030 μg/μl to 0.08 μg/μl, 0.040 μg/μl to 0.08 μg/μl, 0.050 μg/μl to 0.08 μg/μl, 0.060 μg/μl to 0.08 μg/μl, 0.070 μg/μl to 0.08 μg/μl, 0.020 μg/μl to 0.07 μg/μl, 0.030 μg/μl to 0.07 μg/μl, 0.040 μg/μl to 0.07 μg/μl, 0.050 μg/μl to 0.07 μg/μl, 0.060 μg/μl to 0.07 μg/μl, 0.020 μg/μl to 0.06 μg/μl, 0.030 μg/μl to 0.06 μg/μl, 0.040 μg/μl to 0.06 μg/μl, 0.050 μg/μl to 0.06 μg/μl, 0.020 μg/μl to 0.05 μg/μl, 0.030 μg/μl to 0.05 μg/μl, 0.040 μg/μl to 0.05 μg/μl, 0.020 μg/μl to 0.04 μg/μl, 0.030 μg/μl to 0.04 μg/μl, and 0.020 μg/μl to 0.03 μg/μl DNA template. In embodiments, the in vitro reaction mixture comprises 0.020 μg/μl, 0.030 μg/μl, 0.040 μg/μl, 0.050 μg/μl, 0.060 μg/μl, 0.070 μg/μl, 0.080 μg/μl, 0.090 μg/μl or 0.1 μg/μl DNA template.

In embodiments, the DNA template can be, for example, a linearized plasmid or PCR product. In embodiments, the DNA template is a linearized plasmid. In embodiments, the DNA template is a PCR product.

In embodiments, where the DNA template is a linearized plasmid the concentration of the DNA template in the reaction mixture is 0.050 μg/μl. In embodiments, where the DNA template is a PCR product the concentration of the DNA template in the reaction mixture is 0.025 μg/μl.

In embodiments, the DNA template is 0.3 kb to 10 kb in size. For example, the size of the DNA template can be selected from 0.3 kb to 10 kb, 0.4 kb to 10 kb, 0.5 kb to 10 kb, 0.6 kb to 10 kb, 0.7 kb to 10 kb, 0.8 kb to 10 kb, 0.9 kb to 10 kb, 1 kb to 10 kb, 1.1 kb to 10 kb, 1.2 kb to 10 kb, 1.3 kb to 10 kb, 1.4 kb to 10 kb, 1.5 kb to 10 kb, 1.6 kb to 10 kb, 1.7 kb to 10 kb, 1.8 kb to 10 kb, 1.9 kb to 10 kb, 2 kb to 10 kb, 2.1 kb to 10 kb, 2.2 kb to 10 kb, 2.3 kb to 10 kb, 2.4 kb to 10 kb, 2.5 kb to 10 kb, 2.6 kb to 10 kb, 2.7 kb to 10 kb, 2.8 kb to 10 kb, 2.9 kb to 10 kb, 3 kb to 10 kb, 3.1 kb to 10 kb, 3.2 kb to 10 kb, 3.3 kb to 10 kb, 3.4 kb to 10 kb, 3.5 kb to 10 kb, 3.6 kb to 10 kb, 3.7 kb to 10 kb, 3.8 kb to 10 kb, 3.9 kb to 10 kb, 4 kb to 10 kb, 4.1 kb to 10 kb, 4.2 kb to 10 kb, 4.3 kb to 10 kb, 4.4 kb to 10 kb, 4.5 kb to 10 kb, 4.6 kb to 10 kb, 4.7 kb to 10 kb, 4.8 kb to 10 kb, 4.9 kb to 10 kb, 5 kb to 10 kb, 5.1 kb to 10 kb, 5.2 kb to 10 kb, 5.3 kb to 10 kb, 5.4 kb to 10 kb, 5.5 kb to 10 kb, 5.6 kb to 10 kb, 5.7 kb to 10 kb, 5.8 kb to 10 kb, 5.9 kb to 10 kb, 6 kb to 10 kb, 6.1 kb to 10 kb, 6.2 kb to 10 kb, 6.3 kb to 10 kb, 6.4 kb to 10 kb, 6.5 kb to 10 kb, 6.6 kb to 10 kb, 6.7 kb to 10 kb, 6.8 kb to 10 kb, 6.9 kb to 10 kb, 7 kb to 10 kb, 7.1 kb to 10 kb, 7.2 kb to 10 kb, 7.3 kb to 10 kb, 7.4 kb to 10 kb, 7.5 kb to 10 kb, 7.6 kb to 10 kb, 7.7 kb to 10 kb, 7.8 kb to 10 kb, 7.9 kb to 10 kb, 8.0 kb to 10 kb, 8.1 kb to 10 kb, 8.2 kb to 10 kb, 8.3 kb to 10 kb, 8.4 kb to 10 kb, 8.5 kb to 10 kb, 8.6 kb to 10 kb, 8.7 kb to 10 kb, 8.8 kb to 10 kb, 8.9 kb to 10 kb, 9.0 kb to 10 kb, 9.1 kb to 10 kb, 9.2 kb to 10 kb, 9.3 kb to 10 kb, 9.4 kb to 10 kb, 9.5 kb to 10 kb, 9.6 kb to 10 kb, 9.7 kb to 10 kb, 9.8 kb to 10 kb, and 9.9 kb to 10 kb. In embodiments, the DNA template is 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10 kb in length.

In embodiments, the reaction mixture comprises NTPs, including ATP, CTP, GTP and UTP. In embodiments, one or more of the NTPs is a modified NTP. Exemplary modified bases include, but are not limited to, inosine, 7-deazaguanosine, 7-methylguanosine, dihyrouridine, 2′-O-methylguanosine, 2′-fluoro-2′-deoxycytidine, pseudouridine, N1-methylpseudouridine, and 5-methyluridine. In embodiments, one or more uridines in the in vitro transcribed mRNA are replaced by a modified nucleoside.

In embodiments, 3 mM to 7 mM of ATP, 3 mM to 7 mM CTP, 3 mM to 7 GTP, and 3 mM to 7 UTP are included in the reaction mixture. For example, the reaction mixture can comprise a concentration of ATP, CTP, GTP or UTP selected from 3 mM to 7 mM, 3.1 mM to 7 mM, 3.2 mM to 7 mM, 3.3 mM to 7 mM, 3.4 mM to 7 mM, 3.5 mM to 7 mM, 3.6 mM to 7 mM, 3.7 mM to 7 mM, 3.8 mM to 7 mM, 3.9 mM to 7 mM, 4.0 mM to 7 mM, 4.1 mM to 7 mM, 4.2 mM to 7 mM, 4.3 mM to 7 mM, 4.4 mM to 7 mM, 4.5 mM to 7 mM, 4.6 mM to 7 mM, 4.7 mM to 7 mM, 4.8 mM to 7 mM, 4.9 mM to 7 mM, 5.0 mM to 7 mM, 5.1 mM to 7 mM, 5.2 mM to 7 mM, 5.3 mM to 7 mM, 5.4 mM to 7 mM, 5.5 mM to 7 mM, 5.6 mM to 7 mM, 5.7 mM to 7 mM, 5.8 mM to 7 mM, 5.9 mM to 7 mM, 6.0 mM to 7 mM, 6.1 mM to 7 mM, 6.2 mM to 7 mM, 6.3 mM to 7 mM, 6.4 mM to 7 mM, 6.5 mM to 7 mM, 6.6 mM to 7 mM, 6.7 mM to 7 mM, 6.8 mM to 7 mM, 6.9 mM to 7 mM, 3 mM to 6 mM, 3.1 mM to 6 mM, 3.2 mM to 6 mM, 3.3 mM to 6 mM, 3.4 mM to 6 mM, 3.5 mM to 6 mM, 3.6 mM to 6 mM, 3.7 mM to 6 mM, 3.8 mM to 6 mM, 3.9 mM to 6 mM, 4.0 mM to 6 mM, 4.1 mM to 6 mM, 4.2 mM to 6 mM, 4.3 mM to 6 mM, 4.4 mM to 6 mM, 4.5 mM to 6 mM, 4.6 mM to 6 mM, 4.7 mM to 6 mM, 4.8 mM to 6 mM, 4.9 mM to 6 mM, 5.0 mM to 6 mM, 5.1 mM to 6 mM, 5.2 mM to 6 mM, 5.3 mM to 6 mM, 5.4 mM to 6 mM, 5.5 mM to 6 mM, 5.6 mM to 6 mM, 5.7 mM to 6 mM, 5.8 mM to 6 mM, 5.9 mM to 6 mM, 3 mM to 5 mM, 3.1 mM to 5 mM, 3.2 mM to 5 mM, 3.3 mM to 5 mM, 3.4 mM to 5 mM, 3.5 mM to 5 mM, 3.6 mM to 5 mM, 3.7 mM to 5 mM, 3.8 mM to 5 mM, 3.9 mM to 5 mM, 4.0 mM to 5 mM, 4.1 mM to 5 mM, 4.2 mM to 5 mM, 4.3 mM to 5 mM, 4.4 mM to 5 mM, 4.5 mM to 5 mM, 4.6 mM to 5 mM, 4.7 mM to 5 mM, 4.8 mM to 5 mM, 4.9 mM to 5 mM, 3 mM to 4 mM, 3.1 mM to 4 mM, 3.2 mM to 4 mM, 3.3 mM to 4 mM, 3.4 mM to 4 mM, 3.5 mM to 4 mM, 3.6 mM to 4 mM, 3.7 mM to 4 mM, 3.8 mM to 4 mM, and 3.9 mM to 4 mM. In embodiments, the concentration of each NTP in the reaction mixture is selected from 3 mM, 3.1 mM, 3.2 mM, 3.3 mM, 3.4 mM, 3.5 mM, 3.6 mM, 3.7 mM, 3.8 mM, 3.9 mM, 4.0 mM, 4.1 mM, 4.2 mM, 4.3 mM, 4.4 mM, 4.5 mM, 4.6 mM, 4.7 mM, 4.8 mM, 4.9 mM, 5.0 mM, 5.1 mM, 5.2 mM, 5.3 mM, 5.4 mM, 5.5 mM, 5.6 mM, 5.7 mM, 5.8 mM, 5.9 mM, 6.0 mM, 6.1 mM, 6.2 mM, 6.3 mM, 6.4 mM, 6.5 mM, 6.6 mM, 6.7 mM, 6.8 mM, 6.9 mM, and 7.0 mM.

In embodiments, the Spike-in mix comprises NTPs, including ATP, CTP, GTP and UTP. In embodiments, one or more of the NTPs is a modified NTP. Exemplary modified bases include, but are not limited to, inosine, 7-deazaguanosine, 7-methylguanosine, dihyrouridine, 2′-O-methylguanosine, 2′-fluoro-2′-deoxycytidine, pseudouridine, N1-methylpseudouridine, and 5-methyluridine. In embodiments, one or more uridines in the in vitro transcribed mRNA are replaced by a modified nucleoside.

In embodiments, 3 mM to 8 mM of ATP, 3 mM to 8 mM CTP, 3 mM to 8 GTP, and 3 mM to 8 UTP are included in the Spike-in mix. For example, the Spike-in mix can comprise a concentration of ATP, CTP, GTP or UTP selected from 3 mM to 8 mM, 3.1 mM to 8 mM, 3.2 mM to 8 mM, 3.3 mM to 8 mM, 3.4 mM to 8 mM, 3.5 mM to 8 mM, 3.6 mM to 8 mM, 3.7 mM to 8 mM, 3.8 mM to 8 mM, 3.9 mM to 8 mM, 4.0 mM to 8 mM, 4.1 mM to 8 mM, 4.2 mM to 8 mM, 4.3 mM to 8 mM, 4.4 mM to 8 mM, 4.5 mM to 8 mM, 4.6 mM to 8 mM, 4.7 mM to 8 mM, 4.8 mM to 8 mM, 4.9 mM to 8 mM, 5.0 mM to 8 mM, 5.1 mM to 8 mM, 5.2 mM to 8 mM, 5.3 mM to 8 mM, 5.4 mM to 8 mM, 5.5 mM to 8 mM, 5.6 mM to 8 mM, 5.7 mM to 8 mM, 5.8 mM to 8 mM, 5.9 mM to 8 mM, 6.0 mM to 8 mM, 6.1 mM to 8 mM, 6.2 mM to 8 mM, 6.3 mM to 8 mM, 6.4 mM to 8 mM, 6.5 mM to 8 mM, 6.6 mM to 8 mM, 6.7 mM to 8 mM, 6.8 mM to 8 mM, 6.9 mM to 8 mM, 7.0 mM to 8 mM, 7.1 mM to 8 mM, 7.2 mM to 8 mM, 7.3 mM to 8 mM, 7.4 mM to 8 mM, 7.5 mM to 8 mM, 7.6 mM to 8 mM, 7.7 mM to 8 mM, 7.8 mM to 8 mM, 7.9 mM to 8 mM, 3 mM to 7 mM, 3.1 mM to 7 mM, 3.2 mM to 7 mM, 3.3 mM to 7 mM, 3.4 mM to 7 mM, 3.5 mM to 7 mM, 3.6 mM to 7 mM, 3.7 mM to 7 mM, 3.8 mM to 7 mM, 3.9 mM to 7 mM, 4.0 mM to 7 mM, 4.1 mM to 7 mM, 4.2 mM to 7 mM, 4.3 mM to 7 mM, 4.4 mM to 7 mM, 4.5 mM to 7 mM, 4.6 mM to 7 mM, 4.7 mM to 7 mM, 4.8 mM to 7 mM, 4.9 mM to 7 mM, 5.0 mM to 7 mM, 5.1 mM to 7 mM, 5.2 mM to 7 mM, 5.3 mM to 7 mM, 5.4 mM to 7 mM, 5.5 mM to 7 mM, 5.6 mM to 7 mM, 5.7 mM to 7 mM, 5.8 mM to 7 mM, 5.9 mM to 7 mM, 6.0 mM to 7 mM, 6.1 mM to 7 mM, 6.2 mM to 7 mM, 6.3 mM to 7 mM, 6.4 mM to 7 mM, 6.5 mM to 7 mM, 6.6 mM to 7 mM, 6.7 mM to 7 mM, 6.8 mM to 7 mM, 6.9 mM to 7 mM, 3 mM to 6 mM, 3.1 mM to 6 mM, 3.2 mM to 6 mM, 3.3 mM to 6 mM, 3.4 mM to 6 mM, 3.5 mM to 6 mM, 3.6 mM to 6 mM, 3.7 mM to 6 mM, 3.8 mM to 6 mM, 3.9 mM to 6 mM, 4.0 mM to 6 mM, 4.1 mM to 6 mM, 4.2 mM to 6 mM, 4.3 mM to 6 mM, 4.4 mM to 6 mM, 4.5 mM to 6 mM, 4.6 mM to 6 mM, 4.7 mM to 6 mM, 4.8 mM to 6 mM, 4.9 mM to 6 mM, 5.0 mM to 6 mM, 5.1 mM to 6 mM, 5.2 mM to 6 mM, 5.3 mM to 6 mM, 5.4 mM to 6 mM, 5.5 mM to 6 mM, 5.6 mM to 6 mM, 5.7 mM to 6 mM, 5.8 mM to 6 mM, 5.9 mM to 6 mM, 3 mM to 5 mM, 3.1 mM to 5 mM, 3.2 mM to 5 mM, 3.3 mM to 5 mM, 3.4 mM to 5 mM, 3.5 mM to 5 mM, 3.6 mM to 5 mM, 3.7 mM to 5 mM, 3.8 mM to 5 mM, 3.9 mM to 5 mM, 4.0 mM to 5 mM, 4.1 mM to 5 mM, 4.2 mM to 5 mM, 4.3 mM to 5 mM, 4.4 mM to 5 mM, 4.5 mM to 5 mM, 4.6 mM to 5 mM, 4.7 mM to 5 mM, 4.8 mM to 5 mM, 4.9 mM to 5 mM, 3 mM to 4 mM, 3.1 mM to 4 mM, 3.2 mM to 4 mM, 3.3 mM to 4 mM, 3.4 mM to 4 mM, 3.5 mM to 4 mM, 3.6 mM to 4 mM, 3.7 mM to 4 mM, 3.8 mM to 4 mM, and 3.9 mM to 4 mM. In embodiments, the concentration of each NTP in the Spike-in mix is selected from 3 mM, 3.1 mM, 3.2 mM, 3.3 mM, 3.4 mM, 3.5 mM, 3.6 mM, 3.7 mM, 3.8 mM, 3.9 mM, 4.0 mM, 4.1 mM, 4.2 mM, 4.3 mM, 4.4 mM, 4.5 mM, 4.6 mM, 4.7 mM, 4.8 mM, 4.9 mM, 5.0 mM, 5.1 mM, 5.2 mM, 5.3 mM, 5.4 mM, 5.5 mM, 5.6 mM, 5.7 mM, 5.8 mM, 5.9 mM, 6.0 mM, 6.1 mM, 6.2 mM, 6.3 mM, 6.4 mM, 6.5 mM, 6.6 mM, 6.7 mM, 6.8 mM, 6.9 mM, 7.0 mM, 7.1 mM, 7.2 mM, 7.3 mM, 7.4 mM, 7.5 mM, 7.6 mM, 7.7 mM, 7.8 mM, 7.9 mM, and 8.0 mM.

In embodiments, the concentration of each NTP in the Final Reaction Mixture is selected from 3 mM to 8 mM of ATP, 3 mM to 8 mM CTP, 3 mM to 8 GTP, and 3 mM to 8 UTP. In embodiments, the concentration of the Final Reaction Mixture (which includes the reaction mixture after Spike-in was added) can comprise a concentration of ATP, CTP, GTP or UTP selected from 3 mM to 8 mM, 3.1 mM to 8 mM, 3.2 mM to 8 mM, 3.3 mM to 8 mM, 3.4 mM to 8 mM, 3.5 mM to 8 mM, 3.6 mM to 8 mM, 3.7 mM to 8 mM, 3.8 mM to 8 mM, 3.9 mM to 8 mM, 4.0 mM to 8 mM, 4.1 mM to 8 mM, 4.2 mM to 8 mM, 4.3 mM to 8 mM, 4.4 mM to 8 mM, 4.5 mM to 8 mM, 4.6 mM to 8 mM, 4.7 mM to 8 mM, 4.8 mM to 8 mM, 4.9 mM to 8 mM, 5.0 mM to 8 mM, 5.1 mM to 8 mM, 5.2 mM to 8 mM, 5.3 mM to 8 mM, 5.4 mM to 8 mM, 5.5 mM to 8 mM, 5.6 mM to 8 mM, 5.7 mM to 8 mM, 5.8 mM to 8 mM, 5.9 mM to 8 mM, 6.0 mM to 8 mM, 6.1 mM to 8 mM, 6.2 mM to 8 mM, 6.3 mM to 8 mM, 6.4 mM to 8 mM, 6.5 mM to 8 mM, 6.6 mM to 8 mM, 6.7 mM to 8 mM, 6.8 mM to 8 mM, 6.9 mM to 8 mM, 7.0 mM to 8 mM, 7.1 mM to 8 mM, 7.2 mM to 8 mM, 7.3 mM to 8 mM, 7.4 mM to 8 mM, 7.5 mM to 8 mM, 7.6 mM to 8 mM, 7.7 mM to 8 mM, 7.8 mM to 8 mM, 7.9 mM to 8 mM, 3 mM to 7 mM, 3.1 mM to 7 mM, 3.2 mM to 7 mM, 3.3 mM to 7 mM, 3.4 mM to 7 mM, 3.5 mM to 7 mM, 3.6 mM to 7 mM, 3.7 mM to 7 mM, 3.8 mM to 7 mM, 3.9 mM to 7 mM, 4.0 mM to 7 mM, 4.1 mM to 7 mM, 4.2 mM to 7 mM, 4.3 mM to 7 mM, 4.4 mM to 7 mM, 4.5 mM to 7 mM, 4.6 mM to 7 mM, 4.7 mM to 7 mM, 4.8 mM to 7 mM, 4.9 mM to 7 mM, 5.0 mM to 7 mM, 5.1 mM to 7 mM, 5.2 mM to 7 mM, 5.3 mM to 7 mM, 5.4 mM to 7 mM, 5.5 mM to 7 mM, 5.6 mM to 7 mM, 5.7 mM to 7 mM, 5.8 mM to 7 mM, 5.9 mM to 7 mM, 6.0 mM to 7 mM, 6.1 mM to 7 mM, 6.2 mM to 7 mM, 6.3 mM to 7 mM, 6.4 mM to 7 mM, 6.5 mM to 7 mM, 6.6 mM to 7 mM, 6.7 mM to 7 mM, 6.8 mM to 7 mM, 6.9 mM to 7 mM, 3 mM to 6 mM, 3.1 mM to 6 mM, 3.2 mM to 6 mM, 3.3 mM to 6 mM, 3.4 mM to 6 mM, 3.5 mM to 6 mM, 3.6 mM to 6 mM, 3.7 mM to 6 mM, 3.8 mM to 6 mM, 3.9 mM to 6 mM, 4.0 mM to 6 mM, 4.1 mM to 6 mM, 4.2 mM to 6 mM, 4.3 mM to 6 mM, 4.4 mM to 6 mM, 4.5 mM to 6 mM, 4.6 mM to 6 mM, 4.7 mM to 6 mM, 4.8 mM to 6 mM, 4.9 mM to 6 mM, 5.0 mM to 6 mM, 5.1 mM to 6 mM, 5.2 mM to 6 mM, 5.3 mM to 6 mM, 5.4 mM to 6 mM, 5.5 mM to 6 mM, 5.6 mM to 6 mM, 5.7 mM to 6 mM, 5.8 mM to 6 mM, 5.9 mM to 6 mM, 3 mM to 5 mM, 3.1 mM to 5 mM, 3.2 mM to 5 mM, 3.3 mM to 5 mM, 3.4 mM to 5 mM, 3.5 mM to 5 mM, 3.6 mM to 5 mM, 3.7 mM to 5 mM, 3.8 mM to 5 mM, 3.9 mM to 5 mM, 4.0 mM to 5 mM, 4.1 mM to 5 mM, 4.2 mM to 5 mM, 4.3 mM to 5 mM, 4.4 mM to 5 mM, 4.5 mM to 5 mM, 4.6 mM to 5 mM, 4.7 mM to 5 mM, 4.8 mM to 5 mM, 4.9 mM to 5 mM, 3 mM to 4 mM, 3.1 mM to 4 mM, 3.2 mM to 4 mM, 3.3 mM to 4 mM, 3.4 mM to 4 mM, 3.5 mM to 4 mM, 3.6 mM to 4 mM, 3.7 mM to 4 mM, 3.8 mM to 4 mM, and 3.9 mM to 4 mM. In embodiments, the concentration of each NTP in the Final Reaction Mixture is selected from 3 mM, 3.1 mM, 3.2 mM, 3.3 mM, 3.4 mM, 3.5 mM, 3.6 mM, 3.7 mM, 3.8 mM, 3.9 mM, 4.0 mM, 4.1 mM, 4.2 mM, 4.3 mM, 4.4 mM, 4.5 mM, 4.6 mM, 4.7 mM, 4.8 mM, 4.9 mM, 5.0 mM, 5.1 mM, 5.2 mM, 5.3 mM, 5.4 mM, 5.5 mM, 5.6 mM, 5.7 mM, 5.8 mM, 5.9 mM, 6.0 mM, 6.1 mM, 6.2 mM, 6.3 mM, 6.4 mM, 6.5 mM, 6.6 mM, 6.7 mM, 6.8 mM, 6.9 mM, 7.0 mM, 7.1 mM, 7.2 mM, 7.3 mM, 7.4 mM, 7.5 mM, 7.6 mM, 7.7 mM, 7.8 mM, 7.9 mM, and 8.0 mM.

In embodiments, 10× Transcription Buffer comprises: (a) about 400 mM Tris-HCl (pH 7.5); (b) about 100 mM dithiothreitol (DTT); (c) about 20 mM spermidine; (d) about 160 mM magnesium chloride; (e) about 150 mM hydrochloric acid; (f) DNase and RNase free water; and wherein the 10× Transcription Buffer has a pH of about 1.5.

In embodiments, the 1× Transcription Buffer is a 1:10 dilution of a 10× Transcription Buffer. In embodiments, the 10× Transcription Buffer is diluted 1:10 in the reaction mixture to a 1× Transcription Buffer concentration.

In embodiments, the reaction mixture comprises 12 mM to 20 mM Mg²⁺. In embodiments, the concentration of Mg²⁺ in the reaction mixture is selected from 12 mM to 20 mM Mg²⁺, 13 mM to 20 mM Mg²⁺, 14 mM to 20 mM Mg²⁺, 15 mM to 20 mM Mg²⁺, 16 mM to 20 mM Mg²⁺, 17 mM to 20 mM Mg²⁺, 18 mM to 20 mM Mg²⁺, 19 mM to 20 mM Mg²⁺, 12 mM to 19 mM Mg²⁺, 13 mM to 19 mM Mg²⁺, 14 mM to 19 mM Mg²⁺, 15 mM to 19 mM Mg²⁺, 16 mM to 19 mM Mg²⁺, 17 mM to 19 mM Mg²⁺, 18 mM to 19 mM Mg²⁺, 12 mM to 18 mM Mg²⁺, 13 mM to 18 mM Mg²⁺, 14 mM to 18 mM Mg²⁺, 15 mM to 18 mM Mg²⁺, 16 mM to 18 mM Mg²⁺, 17 mM to 18 mM Mg²⁺, 12 mM to 17 mM Mg²⁺, 13 mM to 17 mM Mg²⁺, 14 mM to 17 mM Mg²⁺, 15 mM to 17 mM Mg²⁺, 16 mM to 17 mM Mg²⁺, 12 mM to 16 mM Mg²⁺, 13 mM to 16 mM Mg²⁺, 14 mM to 16 mM Mg²⁺, 15 mM to 16 mM Mg²⁺, 12 mM to 15 mM Mg²⁺, 13 mM to 15 mM Mg²⁺, 14 mM to 15 mM Mg²⁺, 12 mM to 14 mM Mg²⁺, 13 mM to 14 mM Mg²⁺, and 12 mM to 13 mM Mg²⁺. In embodiments, the reaction mixture comprises 12 mM, 13 mM, 14 mM, 15 mM, 16 mM, 17 mM, 18 mM, 19 mM, or 20 mM Mg²⁺. Any source of magnesium can be used in the methods provided herein. These include, but are not limited to, magnesium chloride, magnesium acetate, magnesium phosphate, magnesium glutamate, magnesium nitrate, and magnesium sulfate. In embodiments, the source of Mg²⁺ in the reaction mixture is magnesium acetate or magnesium chloride.

In embodiments, the reaction mixture comprises 10 U/μL to 18 U/μL of an RNA polymerase, for example, a T7 polymerase or a variant thereof. In embodiments, the concentration of the RNA polymerase is selected from 10 U/μL to 18 U/μL, 11 U/μL to 18 U/μL, 12 U/μL to 18 U/μL, 13 U/μL to 18 U/μL, 14 U/μL to 18 U/μL, 15 U/μL to 18 U/μL, 16 U/μL to 18 U/μL, 17 U/μL to 18 U/μL, 10 U/μL to 17 U/μL, 11 U/μL to 17 U/μL, 12 U/μL to 17 U/μL, 13 U/μL to 17 U/μL, 14 U/μL to 17 U/μL, 15 U/μL to 17 U/μL, 16 U/μL to 17 U/μL, 10 U/μL to 16 U/μL, 11 U/μL to 16 U/μL, 12 U/μL to 16 U/μL, 13 U/μL to 16 U/μL, 14 U/μL to 16 U/μL, 15 U/μL to 16 U/μL, 10 U/μL to 15 U/μL, 11 U/μL to 15 U/μL, 12 U/μL to 15 U/μL, 13 U/μL to 15 U/μL, 14 U/μL to 15 U/μL, 10 U/μL to 14 U/μL, 11 U/μL to 14 U/μL, 12 U/μL to 14 U/μL, 13 U/μL to 14 U/μL, 10 U/μL to 13 U/μL, 11 U/μL to 13 U/μL, 12 U/μL to 13 U/μL, 10 U/μL to 12 U/μL, 11 U/μL to 12 U/μL, and 10 U/μL to 11. In embodiments, the concentration of RNA polymerase in the reaction mixture is 10 U/μL, 11 U/μL, 12 U/μL, 13 U/μL, 14 U/μL, 15 U/μL, 16 U/μL, 17 U/μL, or 18 U/L.

In embodiments, the pH of the reaction mixture is selected from 6.0 to 8.0, 6.1 to 8.0, 6.2 to 8.0, 6.3 to 8.0, 6.4 to 8.0, 6.5 to 8.0, 6.6 to 8.0, 6.7 to 8.0, 6.8 to 8.0, 6.9 to 8.0, 7.0 to 8.0, 7.1 to 8.0, 7.2 to 8.0, 7.3 to 8.0, 7.4 to 8.0, 7.5 to 8.0, 7.6 to 8.0, 7.7 to 8.0, 7.8 to 8.0, 7.9 to 8.0, 6.0 to 7.5, 6.1 to 7.5, 6.2 to 7.5, 6.3 to 7.5, 6.4 to 7.5, 6.5 to 7.5, 6.6 to 7.5, 6.7 to 7.5, 6.8 to 7.5, 6.9 to 7.5, 7.0 to 7.5, 7.1 to 7.5, 7.2 to 7.5, 7.3 to 7.5, 7.4 to 7.5, 6.0 to 7.0, 6.1 to 7.0, 6.2 to 7.0, 6.3 to 7.0, 6.4 to 7.0, 6.5 to 7.0, 6.6 to 7.0, 6.7 to 7.0, 6.8 to 7.0, 6.9 to 7.0, 6.0 to 6.5, 6.1 to 6.5, 6.2 to 6.5, 6.3 to 6.5, and 6.4 to 6.5. In embodiments, the pH of the reaction mixture is 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0.

In embodiments, the reaction mixture further comprises one or more of an RNase inhibitor and an inorganic pyrophosphatase. In embodiments, the reaction mixture further comprises one or more of an RNase inhibitor or an inorganic pyrophosphatase. In embodiments, the RNase inhibitor is a murine RNase inhibitor.

In embodiments, the concentration of the RNase inhibitor is selected from 0.5 U/μl to 2 U/μl, 0.6 U/μl to 2 U/μl, 0.7 U/μl to 2 U/μl, 0.8 U/μl to 2 U/μl, 0.9 U/μl to 2 U/μl, 1 U/μl to 2 U/μl, 1.1 U/μl to 2 U/μl, 1.2 U/μl to 2 U/μl, 1.3 U/μl to 2 U/μl, 1.4 U/μl to 2 U/μl, 1.5 U/μl to 2 U/μl, 1.6 U/μl to 2 U/μl, 1.7 U/μl to 2 U/μl, 1.8 U/μl to 2 U/μl, and 1.9 U/μl to 2 U/μl. In embodiments, the concentration of the RNase inhibitor is 0.5 U/μl, 0.6 U/μl, 0.7 U/μl, 0.8 U/μl, 0.9 U/μl, 1 U/μl, 1.1 U/μl, 1.2 U/μl, 1.3 U/μl, 1.4 U/μl, 1.5 U/μl, 1.6 U/μl, 1.7 U/μl, 1.8 U/μl, 1.9 U/μl, or 2 U/μl.

In embodiments, the concentration of the inorganic pyrophosphatase is selected from 0.001 U/μl to 0.02 U/μl, 0.002 U/μl to 0.02 U/μl, 0.003 U/μl to 0.02 U/μl, 0.004 U/μl to 0.02 U/μl, 0.005 U/μl to 0.02 U/μl, 0.006 U/μl to 0.02 U/μl, 0.007 U/μl to 0.02 U/μl, 0.008 U/μl to 0.02 U/μl, 0.009 U/μl to 0.02 U/μl, 0.01 U/μl to 0.02 U/μl, 0.011 U/μl to 0.02 U/μl, 0.012 U/μl to 0.02 U/μl, 0.013 U/μl to 0.02 U/μl, 0.014 U/μl to 0.02 U/μl, 0.015 U/μl to 0.02 U/μl, 0.016 U/μl to 0.02 U/μl, 0.017 U/μl to 0.02 U/μl, 0.018 U/μl to 0.02 U/μl, 0.019 U/μl to 0.02 U/μl, 0.002 U/μl to 0.01 U/μl, 0.003 U/μl to 0.01 U/μl, 0.004 U/μl to 0.01 U/μl, 0.005 U/μl to 0.01 U/μl, 0.006 U/μl to 0.01 U/μl, 0.007 U/μl to 0.01 U/μl, 0.008 U/μl to 0.01 U/μl, and 0.009 U/μl to 0.01 U/μl. In embodiments, the concentration of the inorganic pyrophosphatase is 0.001 U/μl, 0.002 U/μl, 0.003 U/μl, 0.004 U/μl, 0.005 U/μl, 0.006 U/μl, 0.007 U/μl, 0.008 U/μl, 0.009 U/μl, 0.01 U/μl, or 0.02 U/μl.

In embodiments, the reaction mixture is incubated at a temperature that is favorable for in vitro RNA transcription by the RNA polymerase, i.e., at 34° C. to 44° C. In some methods, the incubation temperature is selected from 34° C. to 44° C., 35° C. to 44° C., 36° C. to 44° C., 37° C. to 44° C., 38° C. to 44° C., 39° C. to 44° C., 40° C. to 44° C., 41° C. to 44° C., 42° C. to 44° C., 43° C. to 44° C., 34° C. to 43° C., 35° C. to 43° C., 36° C. to 43° C., 37° C. to 43° C., 38° C. to 43° C., 39° C. to 43° C., 40° C. to 43° C., 41° C. to 43° C., 42° C. to 43° C., 34° C. to 42° C., 35° C. to 42° C., 36° C. to 42° C., 37° C. to 42° C., 38° C. to 42° C., 39° C. to 42° C., 40° C. to 42° C., 41° C. to 42° C., 34° C. to 41° C., 35° C. to 41° C., 36° C. to 41° C., 37° C. to 41° C., 38° C. to 41° C., 39° C. to 41° C., 40° C. to 41° C., 34° C. to 40° C., 35° C. to 40° C., 36° C. to 40° C., 37° C. to 40° C., 38° C. to 40° C., 39° C. to 40° C., 34° C. to 39° C., 35° C. to 39° C., 36° C. to 39° C., 37° C. to 39° C., 38° C. to 39° C., 34° C. to 38° C., 35° C. to 38° C., 36° C. to 38° C., 37° C. to 38° C., 34° C. to 37° C., 35° C. to 37° C., and 36° C. to 37° C. In embodiments, the incubation temperature is selected from 34° C., 35° C., 36° C., 37° C., 38° C., 39° C., 40° C., 41° C., 42° C., 43° C. and 44° C.

In embodiments, the reaction mixture is incubated for one hour or longer. In embodiments, the reaction mixture is incubated for greater than 1 hour. In embodiments, the reaction mixture is incubated for 2 to 3 hours. In embodiments, the incubation time is selected from 60 min to 180 min, 65 min to 180 min, 70 min to 180 min, 75 mM to 180 mM, 80 min to 180 min, 85 mM to 180 mM, 90 min to 180 min, 95 min to 180 min, 100 min to 180 min, 105 min, to 180 min, 110 min to 180 min, 115 min to 180 min, 120 min, to 180 min, 125 min to 180 min, 130 min to 180 min, 135 min to 180 min, 140 min to 180 min, 145 min to 180 min, 150 min to 180 min, 155 min to 180 min, 160 min to 180 min, 165 min to 180 min, 170 min to 180 min, 175 min to 180 min, 60 min to 170 min, 65 min to 170 min, 70 min to 170 min, 75 min to 170 min 80 min to 170 min, 85 min to 170 min 90 min to 170 min, 95 min to 170 min 100 min to 170 min, 105 min to 170 min 110 min to 170 min, 115 min to 170 min, 120 min, to 170 min, 125 min to 170 min, 130 min to 170 min, 135 min to 170 min, 140 min to 170 min, 145 min to 170 min, 150 min to 170 min, 155 min to 170 min, 160 min to 170 min, 165 min to 170 min, 120 min to 170 min, 125 min to 170 min, 130 min to 170 min, 135 min to 170 min, 140 min to 170 min, 145 min to 170 min, 150 min to 170 min, 155 min to 170 min, 160 min to 170 min, 165 min to 170 min, 60 min to 160 min, 65 min to 160 min, 70 min to 160 min, 75 min to 160 min, 80 min to 160 min, 85 min to 160 min, 90 min to 160 min, 95 min to 160 min, 100 min to 160 min, 105 min to 160 min, 110 min to 160 min, 115 min to 160 min, 120 min to 160 min, 125 min to 160 min, 130 min to 160 min, 135 min to 160 min, 140 min to 160 min, 145 min to 160 min, 150 min to 160 min, 155 min to 160 min, 60 min to 150 min, 65 min to 150 min, 70 min to 150 min, 75 min to 150 min, 80 min to 150 min, 85 min to 150 min, 90 min to 150 min, 95 min to 150 min, 100 min to 150 min, 105 min to 150 min, 110 min to 150 min, 115 min to 150 min, 120 min to 150 min, 125 min to 150 min, 130 min to 150 min, 135 min to 150 min, 140 min to 150 min, 145 min to 150 min, 120 min to 150 min, 125 min to 150 min, 130 min to 150 min, 135 min to 150 min, 140 min to 150 min, 145 min to 150 min, 60 min to 140 min, 65 min to 140 min, 70 min to 140 min, 75 min to 140 min, 80 min to 140 min, 85 min to 140 min, 90 min to 140 min, 95 min to 140 min, 100 min to 140 min, 105 min to 140 min, 110 min to 140 min, 115 min to 140 min, 120 min to 140 min, 125 min to 140 min, 130 min to 140 min, 135 min to 140 min, 120 min to 140 min, 125 min to 140 min, 130 min to 140 min, 135 min to 140 min, 60 min to 130 min, 65 min to 130 min, 70 min to 130 min, 75 min to 130 min, 80 min to 130 min, 85 min to 130 min, 90 min to 130 min, 95 min to 130 min, 100 min to 130 min, 105 min to 130 min, 110 min to 130 min, 115 min to 130 min, 120 min to 130 min, and 125 min to 130 min. In embodiments, the incubation time is selected from 60 min, 65 min, 70 min, 75 min, 80 min, 90 min, 95 min, 100 min, 105 min, 110 min, 115 min, 120 min, 125 min, 130 min, 135 min, 140 min, 145 min, 150 min, 155 min, 160 min, 165 min, 170 min, and 180 min.

In embodiments, Spike-in is added to the solution comprising capped mRNA transcripts and the Final Reaction Mixture is incubated for 2 to 3 hours. In embodiments, the incubation time is selected from 60 min to 180 min, 65 min to 180 min, 70 min to 180 min, 75 mM to 180 mM, 80 min to 180 min, 85 mM to 180 mM, 90 min to 180 min, 95 min to 180 min, 100 min to 180 min, 105 min, to 180 min, 110 min to 180 min, 115 min to 180 min, 120 min, to 180 min, 125 min to 180 min, 130 min to 180 min, 135 min to 180 min, 140 min to 180 min, 145 min to 180 min, 150 min to 180 min, 155 min to 180 min, 160 min to 180 min, 165 min to 180 min, 170 min to 180 min, 175 min to 180 min, 60 min to 170 min, 65 min to 170 min, 70 min to 170 min, 75 min to 170 min 80 min to 170 min, 85 min to 170 min 90 min to 170 min, 95 min to 170 min 100 min to 170 min, 105 min to 170 min 110 min to 170 min, 115 min to 170 min, 120 min, to 170 min, 125 min to 170 min, 130 min to 170 min, 135 min to 170 min, 140 min to 170 min, 145 min to 170 min, 150 min to 170 min, 155 min to 170 min, 160 min to 170 min, 165 min to 170 min, 120 min to 170 min, 125 min to 170 min, 130 min to 170 min, 135 min to 170 min, 140 min to 170 min, 145 min to 170 min, 150 min to 170 min, 155 min to 170 min, 160 min to 170 min, 165 min to 170 min, 60 min to 160 min, 65 min to 160 min, 70 min to 160 min, 75 min to 160 min, 80 min to 160 min, 85 min to 160 min, 90 min to 160 min, 95 min to 160 min, 100 min to 160 min, 105 min to 160 min, 110 min to 160 min, 115 min to 160 min, 120 min to 160 min, 125 min to 160 min, 130 min to 160 min, 135 min to 160 min, 140 min to 160 min, 145 min to 160 min, 150 min to 160 min, 155 min to 160 min, 60 min to 150 min, 65 min to 150 min, 70 min to 150 min, 75 min to 150 min, 80 min to 150 min, 85 min to 150 min, 90 min to 150 min, 95 min to 150 min, 100 min to 150 min, 105 min to 150 min, 110 min to 150 min, 115 min to 150 min, 120 min to 150 min, 125 min to 150 min, 130 min to 150 min, 135 min to 150 min, 140 min to 150 min, 145 min to 150 min, 120 min to 150 min, 125 min to 150 min, 130 min to 150 min, 135 min to 150 min, 140 min to 150 min, 145 min to 150 min, 60 min to 140 min, 65 min to 140 min, 70 min to 140 min, 75 min to 140 min, 80 min to 140 min, 85 min to 140 min, 90 min to 140 min, 95 min to 140 min, 100 min to 140 min, 105 min to 140 min, 110 min to 140 min, 115 min to 140 min, 120 min to 140 min, 125 min to 140 min, 130 min to 140 min, 135 min to 140 min, 120 min to 140 min, 125 min to 140 min, 130 min to 140 min, 135 min to 140 min, 60 min to 130 min, 65 min to 130 min, 70 min to 130 min, 75 min to 130 min, 80 min to 130 min, 85 min to 130 min, 90 min to 130 min, 95 min to 130 min, 100 min to 130 min, 105 min to 130 min, 110 min to 130 min, 115 min to 130 min, 120 min to 130 min, and 125 min to 130 min. In embodiments, the incubation time is selected from 60 min, 65 min, 70 min, 75 min, 80 min, 90 min, 95 min, 100 min, 105 min, 110 min, 115 min, 120 min, 125 min, 130 min, 135 min, 140 min, 145 min, 150 min, 155 min, 160 min, 165 min, 170 min, and 180 min.

In embodiments, the method further comprises incubating the solution comprising capped mRNA transcripts or the Final Reaction Mixture with: (i) a solution of CaCl₂) with a concentration of 0.5 mM to 5 mM in the reaction mixture or the Final Reaction Mixture; and (ii) DNase I. In embodiments, the concentration of Ca²⁺ in the solution is selected from, 0.5 mM to 5.0 mM, 0.6 mM to 5.0 mM, 0.7 mM to 5.0 mM, 0.8 mM to 5.0 mM, 0.9 mM to 5.0 mM, 1.0 mM to 5.0 mM, 1.1 mM to 5.0 mM, 1.2 mM to 5.0 mM, 1.3 mM to 5.0 mM, 1.4 mM to 5.0 mM, 1.5 mM to 4.0 mM, 1.6 mM to 5.0 mM, 1.7 mM to 5.0 mM, 1.8 mM to 5.0 mM, 1.9 mM to 5.0 mM, 2.0 mM to 5.0 mM, 2.1 mM to 5.0 mM, 2.2 mM to 5.0 mM, 2.3 mM to 5.0 mM, 2.4 mM to 5.0 mM, 2.5 mM to 5.0 mM, 2.6 mM to 5.0 mM, 2.7 mM to 5.0 mM, 2.8 mM to 5.0 mM, 2.9 mM to 5.0 mM, 3.0 mM to 5.0 mM, 3.1 mM to 5.0 mM, 3.2 mM to 5.0 mM, 3.3 mM to 5.0 mM, 3.4 mM to 5.0 mM, 3.5 mM to 5.0 mM, 3.6 mM to 5.0 mM, 3.7 mM to 5.0 mM, 3.8 mM to 5.0 mM, 3.9 mM to 5.0 mM, 4.0 mM to 5.0 mM, 4.1 mM to 5.0 mM, 4.2 mM to 5.0 mM, 4.3 mM to 5.0 mM, 4.4 mM to 5.0 mM, 4.5 mM to 5.0 mM, 4.6 mM to 5.0 mM, 4.7 mM to 5.0 mM, 4.8 mM to 5.0 mM, 4.9 mM to 5.0 mM, 0.5 mM to 4.0 mM, 0.6 mM to 4.0 mM, 0.7 mM to 4.0 mM, 0.8 mM to 4.0 mM, 0.9 mM to 4.0 mM, 1.0 mM to 4.0 mM, 1.1 mM to 4.0 mM, 1.2 mM to 4.0 mM, 1.3 mM to 4.0 mM, 1.4 mM to 4.0 mM, 1.5 mM to 4.0 mM, 1.6 mM to 4.0 mM, 1.7 mM to 4.0 mM, 1.8 mM to 4.0 mM, 1.9 mM to 4.0 mM, 2.0 mM to 4.0 mM, 2.1 mM to 4.0 mM, 2.2 mM to 4.0 mM, 2.3 mM to 4.0 mM, 2.4 mM to 4.0 mM, 2.5 mM to 4.0 mM, 2.6 mM to 4.0 mM, 2.7 mM to 4.0 mM, 2.8 mM to 4.0 mM, 2.9 mM to 4.0 mM, 3.0 mM to 4.0 mM, 3.1 mM to 4.0 mM, 3.2 mM to 4.0 mM, 3.3 mM to 4.0 mM, 3.4 mM to 4.0 mM, 3.5 mM to 4.0 mM, 3.6 mM to 4.0 mM, 3.7 mM to 4.0 mM, 3.8 mM to 4.0 mM, 3.9 mM to 4.0 mM, 0.5 mM to 3.9 mM, 0.6 mM to 3.9 mM, 0.7 mM to 3.9 mM, 0.8 mM to 3.9 mM, 0.9 mM to 3.9 mM, 1.0 mM to 3.9 mM, 1.1 mM to 3.9 mM, 1.2 mM to 3.9 mM, 1.3 mM to 3.9 mM, 1.4 mM to 3.9 mM, 1.5 mM to 3.9 mM, 1.6 mM to 3.9 mM, 1.7 mM to 3.9 mM, 1.8 mM to 3.9 mM, 1.9 mM to 3.9 mM, 2.0 mM to 3.9 mM, 2.1 mM to 3.9 mM, 2.2 mM to 3.9 mM, 2.3 mM to 3.9 mM, 2.4 mM to 3.9 mM, 2.5 mM to 3.9 mM, 2.6 mM to 3.9 mM, 2.7 mM to 3.9 mM, 2.8 mM to 3.9 mM, 2.9 mM to 3.9 mM, 3.0 mM to 3.9 mM, 3.1 mM to 3.9 mM, 3.2 mM to 3.9 mM, 3.3 mM to 3.9 mM, 3.4 mM to 3.9 mM, 3.5 mM to 3.9 mM, 3.6 mM to 3.9 mM, 3.7 mM to 3.9 mM, 3.8 mM to 3.9 mM, 0.5 mM to 3.8 mM, 0.6 mM to 3.8 mM, 0.7 mM to 3.8 mM, 0.8 mM to 3.8 mM, 0.9 mM to 3.8 mM, 1.0 mM to 3.8 mM, 1.1 mM to 3.8 mM, 1.2 mM to 3.8 mM, 1.3 mM to 3.8 mM, 1.4 mM to 3.8 mM, 1.5 mM to 3.8 mM, 1.6 mM to 3.8 mM, 1.7 mM to 3.8 mM, 1.8 mM to 3.8 mM, 1.9 mM to 3.8 mM, 2.0 mM to 3.8 mM, 2.1 mM to 3.8 mM, 2.2 mM to 3.8 mM, 2.3 mM to 3.8 mM, 2.4 mM to 3.8 mM, 2.5 mM to 3.8 mM, 2.6 mM to 3.8 mM, 2.7 mM to 3.8 mM, 2.8 mM to 3.8 mM, 2.9 mM to 3.8 mM, 3.0 mM to 3.8 mM, 3.1 mM to 3.8 mM, 3.2 mM to 3.8 mM, 3.3 mM to 3.8 mM, 3.4 mM to 3.8 mM, 3.5 mM to 3.8 mM, 3.6 mM to 3.8 mM, 3.7 mM to 3.8 mM, 0.5 mM to 3.7 mM, 0.6 mM to 3.7 mM, 0.7 mM to 3.7 mM, 0.8 mM to 3.7 mM, 0.9 mM to 3.7 mM, 1.0 mM to 3.7 mM, 1.1 mM to 3.7 mM, 1.2 mM to 3.7 mM, 1.3 mM to 3.7 mM, 1.4 mM to 3.7 mM, 1.5 mM to 3.7 mM, 1.6 mM to 3.7 mM, 1.7 mM to 3.7 mM, 1.8 mM to 3.7 mM, 1.9 mM to 3.7 mM, 2.0 mM to 3.7 mM, 2.1 mM to 3.7 mM, 2.2 mM to 3.7 mM, 2.3 mM to 3.7 mM, 2.4 mM to 3.7 mM, 2.5 mM to 3.7 mM, 2.6 mM to 3.7 mM, 2.7 mM to 3.7 mM, 2.8 mM to 3.7 mM, 2.9 mM to 3.7 mM, 3.0 mM to 3.7 mM, 3.1 mM to 3.7 mM, 3.2 mM to 3.7 mM, 3.3 mM to 3.7 mM, 3.4 mM to 3.7 mM, 3.5 mM to 3.7 mM, 3.6 mM to 3.7 mM, 0.5 mM to 3.6 mM, 0.6 mM to 3.6 mM, 0.7 mM to 3.6 mM, 0.8 mM to 3.6 mM, 0.9 mM to 3.6 mM, 1.0 mM to 3.6 mM, 1.1 mM to 3.6 mM, 1.2 mM to 3.6 mM, 1.3 mM to 3.6 mM, 1.4 mM to 3.6 mM, 1.5 mM to 3.6 mM, 1.6 mM to 3.6 mM, 1.7 mM to 3.6 mM, 1.8 mM to 3.6 mM, 1.9 mM to 3.6 mM, 2.0 mM to 3.6 mM, 2.1 mM to 3.6 mM, 2.2 mM to 3.6 mM, 2.3 mM to 3.6 mM, 2.4 mM to 3.6 mM, 2.5 mM to 3.6 mM, 2.6 mM to 3.6 mM, 2.7 mM to 3.6 mM, 2.8 mM to 3.6 mM, 2.9 mM to 3.6 mM, 3.0 mM to 3.6 mM, 3.1 mM to 3.6 mM, 3.2 mM to 3.6 mM, 3.3 mM to 3.6 mM, 3.4 mM to 3.6 mM, 3.5 mM to 3.6, 0.5 mM to 3.5 mM, 0.6 mM to 3.5 mM, 0.7 mM to 3.5 mM, 0.8 mM to 3.5 mM, 0.9 mM to 3.5 mM, 1.0 mM to 3.5 mM, 1.1 mM to 3.5 mM, 1.2 mM to 3.5 mM, 1.3 mM to 3.5 mM, 1.4 mM to 3.5 mM, 1.5 mM to 3.5 mM, 1.6 mM to 3.5 mM, 1.7 mM to 3.5 mM, 1.8 mM to 3.5 mM, 1.9 mM to 3.5 mM, 2.0 mM to 3.5 mM, 2.1 mM to 3.5 mM, 2.2 mM to 3.5 mM, 2.3 mM to 3.5 mM, 2.4 mM to 3.5 mM, 2.5 mM to 3.5 mM, 2.6 mM to 3.5 mM, 2.7 mM to 3.5 mM, 2.8 mM to 3.5 mM, 2.9 mM to 3.5 mM, 3.0 mM to 3.5 mM, 3.1 mM to 3.5 mM, 3.2 mM to 3.5 mM, 3.3 mM to 3.5 mM, 3.4 mM to 3.5 mM, 0.5 mM to 3.4 mM, 0.6 mM to 3.4 mM, 0.7 mM to 3.4 mM, 0.8 mM to 3.4 mM, 0.9 mM to 3.4 mM, 1.0 mM to 3.4 mM, 1.1 mM to 3.4 mM, 1.2 mM to 3.4 mM, 1.3 mM to 3.4 mM, 1.4 mM to 3.4 mM, 1.5 mM to 3.4 mM, 1.6 mM to 3.4 mM, 1.7 mM to 3.4 mM, 1.8 mM to 3.4 mM, 1.9 mM to 3.4 mM, 2.0 mM to 3.4 mM, 2.1 mM to 3.4 mM, 2.2 mM to 3.4 mM, 2.3 mM to 3.4 mM, 2.4 mM to 3.4 mM, 2.5 mM to 3.4 mM, 2.6 mM to 3.4 mM, 2.7 mM to 3.4 mM, 2.8 mM to 3.4 mM, 2.9 mM to 3.4 mM, 3.0 mM to 3.4 mM, 3.1 mM to 3.4 mM, 3.2 mM to 3.4 mM, 3.3 mM to 3.4 mM, 0.5 mM to 3.3 mM, 0.6 mM to 3.3 mM, 0.7 mM to 3.3 mM, 0.8 mM to 3.3 mM, 0.9 mM to 3.3 mM, 1.0 mM to 3.3 mM, 1.1 mM to 3.3 mM, 1.2 mM to 3.3 mM, 1.3 mM to 3.3 mM, 1.4 mM to 3.3 mM, 1.5 mM to 3.3 mM, 1.6 mM to 3.3 mM, 1.7 mM to 3.3 mM, 1.8 mM to 3.3 mM, 1.9 mM to 3.3 mM, 2.0 mM to 3.3 mM, 2.1 mM to 3.3 mM, 2.2 mM to 3.3 mM, 2.3 mM to 3.3 mM, 2.4 mM to 3.3 mM, 2.5 mM to 3.3 mM, 2.6 mM to 3.3 mM, 2.7 mM to 3.3 mM, 2.8 mM to 3.3 mM, 2.9 mM to 3.3 mM, 3.0 mM to 3.3 mM, 3.1 mM to 3.3 mM, 3.2 mM to 3.3 mM, 0.5 mM to 3.2 mM, 0.6 mM to 3.2 mM, 0.7 mM to 3.2 mM, 0.8 mM to 3.2 mM, 0.9 mM to 3.2 mM, 1.0 mM to 3.2 mM, 1.1 mM to 3.2 mM, 1.2 mM to 3.2 mM, 1.3 mM to 3.2 mM, 1.4 mM to 3.2 mM, 1.5 mM to 3.2 mM, 1.6 mM to 3.2 mM, 1.7 mM to 3.2 mM, 1.8 mM to 3.2 mM, 1.9 mM to 3.2 mM, 2.0 mM to 3.2 mM, 2.1 mM to 3.2 mM, 2.2 mM to 3.2 mM, 2.3 mM to 3.2 mM, 2.4 mM to 3.2 mM, 2.5 mM to 3.2 mM, 2.6 mM to 3.2 mM, 2.7 mM to 3.2 mM, 2.8 mM to 3.2 mM, 2.9 mM to 3.2 mM, 3.0 mM to 3.2 mM, 3.1 mM to 3.2 mM, 0.5 mM to 3.1 mM, 0.6 mM to 3.1 mM, 0.7 mM to 3.1 mM, 0.8 mM to 3.1 mM, 0.9 mM to 3.1 mM, 1.0 mM to 3.1 mM, 1.1 mM to 3.1 mM, 1.2 mM to 3.1 mM, 1.3 mM to 3.1 mM, 1.4 mM to 3.1 mM, 1.5 mM to 3.1 mM, 1.6 mM to 3.1 mM, 1.7 mM to 3.1 mM, 1.8 mM to 3.1 mM, 1.9 mM to 3.1 mM, 2.0 mM to 3.1 mM, 2.1 mM to 3.1 mM, 2.2 mM to 3.1 mM, 2.3 mM to 3.1 mM, 2.4 mM to 3.1 mM, 2.5 mM to 3.1 mM, 2.6 mM to 3.1 mM, 2.7 mM to 3.1 mM, 2.8 mM to 3.1 mM, 2.9 mM to 3.1 mM, 3.0 mM to 3.1 mM, 0.5 mM to 3.0 mM, 0.6 mM to 3.0 mM, 0.7 mM to 3.0 mM, 0.8 mM to 3.0 mM, 0.9 mM to 3.0 mM, 1.0 mM to 3.0 mM, 1.1 mM to 3.0 mM, 1.2 mM to 3.0 mM, 1.3 mM to 3.0 mM, 1.4 mM to 3.0 mM, 1.5 mM to 3.0 mM, 1.6 mM to 3.0 mM, 1.7 mM to 3.0 mM, 1.8 mM to 3.0 mM, 1.9 mM to 3.0 mM, 2.0 mM to 3.0 mM, 2.1 mM to 3.0 mM, 2.2 mM to 3.0 mM, 2.3 mM to 3.0 mM, 2.4 mM to 3.0 mM, 2.5 mM to 3.0 mM, 2.6 mM to 3.0 mM, 2.7 mM to 3.0 mM, 2.8 mM to 3.0 mM, 2.9 mM to 3.0 mM, 0.5 mM to 2.9 mM, 0.6 mM to 2.9 mM, 0.7 mM to 2.9 mM, 0.8 mM to 2.9 mM, 0.9 mM to 2.9 mM, 1.0 mM to 2.9 mM, 1.1 mM to 2.9 mM, 1.2 mM to 2.9 mM, 1.3 mM to 2.9 mM, 1.4 mM to 2.9 mM, 1.5 mM to 2.9 mM, 1.6 mM to 2.9 mM, 1.7 mM to 2.9 mM, 1.8 mM to 2.9 mM, 1.9 mM to 2.9 mM, 2.0 mM to 2.9 mM, 2.1 mM to 2.9 mM, 2.2 mM to 2.9 mM, 2.3 mM to 2.9 mM, 2.4 mM to 2.9 mM, 2.5 mM to 2.9 mM, 2.6 mM to 2.9 mM, 2.7 mM to 2.9 mM, 2.8 mM to 2.9 mM, 0.5 mM to 2.8 mM, 0.6 mM to 2.8 mM, 0.7 mM to 2.8 mM, 0.8 mM to 2.8 mM, 0.9 mM to 2.8 mM, 1.0 mM to 2.8 mM, 1.1 mM to 2.8 mM, 1.2 mM to 2.8 mM, 1.3 mM to 2.8 mM, 1.4 mM to 2.8 mM, 1.5 mM to 2.8 mM, 1.6 mM to 2.8 mM, 1.7 mM to 2.8 mM, 1.8 mM to 2.8 mM, 1.9 mM to 2.8 mM, 2.0 mM to 2.8 mM, 2.1 mM to 2.8 mM, 2.2 mM to 2.8 mM, 2.3 mM to 2.8 mM, 2.4 mM to 2.8 mM, 2.5 mM to 2.8 mM, 2.6 mM to 2.8 mM, 2.7 mM to 2.8 mM, 0.5 mM to 2.7 mM, 0.6 mM to 2.7 mM, 0.7 mM to 2.7 mM, 0.8 mM to 2.7 mM, 0.9 mM to 2.7 mM, 1.0 mM to 2.7 mM, 1.1 mM to 2.7 mM, 1.2 mM to 2.7 mM, 1.3 mM to 2.7 mM, 1.4 mM to 2.7 mM, 1.5 mM to 2.7 mM, 1.6 mM to 2.7 mM, 1.7 mM to 2.7 mM, 1.8 mM to 2.7 mM, 1.9 mM to 2.7 mM, 2.0 mM to 2.7 mM, 2.1 mM to 2.7 mM, 2.2 mM to 2.7 mM, 2.3 mM to 2.7 mM, 2.4 mM to 2.7 mM, 2.5 mM to 2.7 mM, 2.6 mM to 2.7 mM, 0.5 mM to 2.6 mM, 0.6 mM to 2.6 mM, 0.7 mM to 2.6 mM, 0.8 mM to 2.6 mM, 0.9 mM to 2.6 mM, 1.0 mM to 2.6 mM, 1.1 mM to 2.6 mM, 1.2 mM to 2.6 mM, 1.3 mM to 2.6 mM, 1.4 mM to 2.6 mM, 1.5 mM to 2.6 mM, 1.6 mM to 2.6 mM, 1.7 mM to 2.6 mM, 1.8 mM to 2.6 mM, 1.9 mM to 2.6 mM, 2.0 mM to 2.6 mM, 2.1 mM to 2.6 mM, 2.2 mM to 2.6 mM, 2.3 mM to 2.6 mM, 2.4 mM to 2.6 mM, 2.5 mM to 2.6 mM, 0.5 mM to 2.5 mM, 0.6 mM to 2.5 mM, 0.7 mM to 2.5 mM, 0.8 mM to 2.5 mM, 0.9 mM to 2.5 mM, 1.0 mM to 2.5 mM, 1.1 mM to 2.5 mM, 1.2 mM to 2.5 mM, 1.3 mM to 2.5 mM, 1.4 mM to 2.5 mM, 1.5 mM to 2.5 mM, 1.6 mM to 2.5 mM, 1.7 mM to 2.5 mM, 1.8 mM to 2.5 mM, 1.9 mM to 2.5 mM, 2.0 mM to 2.5 mM, 2.1 mM to 2.5 mM, 2.2 mM to 2.5 mM, 2.3 mM to 2.5 mM, 2.4 mM to 2.5 mM, 0.5 mM to 2.4 mM, 0.6 mM to 2.4 mM, 0.7 mM to 2.4 mM, 0.8 mM to 2.4 mM, 0.9 mM to 2.4 mM, 1.0 mM to 2.4 mM, 1.1 mM to 2.4 mM, 1.2 mM to 2.4 mM, 1.3 mM to 2.4 mM, 1.4 mM to 2.4 mM, 1.5 mM to 2.4 mM, 1.6 mM to 2.4 mM, 1.7 mM to 2.4 mM, 1.8 mM to 2.4 mM, 1.9 mM to 2.4 mM, 2.0 mM to 2.4 mM, 2.1 mM to 2.4 mM, 2.2 mM to 2.4 mM, 2.3 mM to 2.4 mM, 0.5 mM to 2.3 mM, 0.6 mM to 2.3 mM, 0.7 mM to 2.3 mM, 0.8 mM to 2.3 mM, 0.9 mM to 2.3 mM, 1.0 mM to 2.3 mM, 1.1 mM to 2.3 mM, 1.2 mM to 2.3 mM, 1.3 mM to 2.3 mM, 1.4 mM to 2.3 mM, 1.5 mM to 2.3 mM, 1.6 mM to 2.3 mM, 1.7 mM to 2.3 mM, 1.8 mM to 2.3 mM, 1.9 mM to 2.3 mM, 2.0 mM to 2.3 mM, 2.1 mM to 2.3 mM, 2.2 mM to 2.3 mM, 0.5 mM to 2.2 mM, 0.6 mM to 2.2 mM, 0.7 mM to 2.2 mM, 0.8 mM to 2.2 mM, 0.9 mM to 2.2 mM, 1.0 mM to 2.2 mM, 1.1 mM to 2.2 mM, 1.2 mM to 2.2 mM, 1.3 mM to 2.2 mM, 1.4 mM to 2.2 mM, 1.5 mM to 2.2 mM, 1.6 mM to 2.2 mM, 1.7 mM to 2.2 mM, 1.8 mM to 2.2 mM, 1.9 mM to 2.2 mM, 2.0 mM to 2.2 mM, 2.1 mM to 2.2 mM, 0.5 mM to 2.1 mM, 0.6 mM to 2.1 mM, 0.7 mM to 2.1 mM, 0.8 mM to 2.1 mM, 0.9 mM to 2.1 mM, 1.0 mM to 2.1 mM, 1.1 mM to 2.1 mM, 1.2 mM to 2.1 mM, 1.3 mM to 2.1 mM, 1.4 mM to 2.1 mM, 1.5 mM to 2.1 mM, 1.6 mM to 2.1 mM, 1.7 mM to 2.1 mM, 1.8 mM to 2.1 mM, 1.9 mM to 2.1 mM, 2.0 mM to 2.1 mM, 0.5 mM to 2.0 mM, 0.6 mM to 2.0 mM, 0.7 mM to 2.0 mM, 0.8 mM to 2.0 mM, 0.9 mM to 2.0 mM, 1.0 mM to 2.0 mM, 1.1 mM to 2.0 mM, 1.2 mM to 2.0 mM, 1.3 mM to 2.0 mM, 1.4 mM to 2.0 mM, 1.5 mM to 2.0 mM, 1.6 mM to 2.0 mM, 1.7 mM to 2.0 mM, 1.8 mM to 2.0 mM, 1.9 mM to 2.0 mM, 0.5 mM to 1.9 mM, 0.6 mM to 1.9 mM, 0.7 mM to 1.9 mM, 0.8 mM to 1.9 mM, 0.9 mM to 1.9 mM, 1.0 mM to 1.9 mM, 1.1 mM to 1.9 mM, 1.2 mM to 1.9 mM, 1.3 mM to 1.9 mM, 1.4 mM to 1.9 mM, 1.5 mM to 1.9 mM, 1.6 mM to 1.9 mM, 1.7 mM to 1.9 mM, 1.8 mM to 1.9 mM, 0.5 mM to 1.8 mM, 0.6 mM to 1.8 mM, 0.7 mM to 1.8 mM, 0.8 mM to 1.8 mM, 0.9 mM to 1.8 mM, 1.0 mM to 1.8 mM, 1.1 mM to 1.8 mM, 1.2 mM to 1.8 mM, 1.3 mM to 1.8 mM, 1.4 mM to 1.8 mM, 1.5 mM to 1.8 mM, 1.6 mM to 1.8 mM, 1.7 mM to 1.8 mM, 0.5 mM to 1.7 mM, 0.6 mM to 1.7 mM, 0.7 mM to 1.7 mM, 0.8 mM to 1.7 mM, 0.9 mM to 1.7 mM, 1.0 mM to 1.7 mM, 1.1 mM to 1.7 mM, 1.2 mM to 1.7 mM, 1.3 mM to 1.7 mM, 1.4 mM to 1.7 mM, 1.5 mM to 1.7 mM, 1.6 mM to 1.7 mM, 0.5 mM to 1.6 mM, 0.6 mM to 1.6 mM, 0.7 mM to 1.6 mM, 0.8 mM to 1.6 mM, 0.9 mM to 1.6 mM, 1.0 mM to 1.6 mM, 1.1 mM to 1.6 mM, 1.2 mM to 1.6 mM, 1.3 mM to 1.6 mM, 1.4 mM to 1.6 mM, 1.5 mM to 1.6 mM, 0.5 mM to 1.5 mM, 0.6 mM to 1.5 mM, 0.7 mM to 1.5 mM, 0.8 mM to 1.5 mM, 0.9 mM to 1.5 mM, 1.0 mM to 1.5 mM, 1.1 mM to 1.5 mM, 1.2 mM to 1.5 mM, 1.3 mM to 1.5 mM, 1.4 mM to 1.5 mM, 0.5 mM to 1.4 mM, 0.6 mM to 1.4 mM, 0.7 mM to 1.4 mM, 0.8 mM to 1.4 mM, 0.9 mM to 1.4 mM, 1.0 mM to 1.4 mM, 1.1 mM to 1.4 mM, 1.2 mM to 1.4 mM, 1.3 mM to 1.4 mM, 0.5 mM to 1.3 mM, 0.6 mM to 1.3 mM, 0.7 mM to 1.3 mM, 0.8 mM to 1.3 mM, 0.9 mM to 1.3 mM, 1.0 mM to 1.3 mM, 1.1 mM to 1.3 mM, 1.2 mM to 1.3 mM, 0.5 mM to 1.2 mM, 0.6 mM to 1.2 mM, 0.7 mM to 1.2 mM, 0.8 mM to 1.2 mM, 0.9 mM to 1.2 mM, 1.0 mM to 1.2 mM, 1.1 mM to 1.2 mM, 0.5 mM to 1.1 mM, 0.6 mM to 1.1 mM, 0.7 mM to 1.1 mM, 0.8 mM to 1.1 mM, 0.9 mM to 1.1 mM, 1.0 mM to 1.1 mM, 0.5 mM to 1.0 mM, 0.6 mM to 1.0 mM, 0.7 mM to 1.0 mM, 0.8 mM to 1.0 mM, 0.9 mM to 1.0 mM, 0.5 mM to 0.9 mM, 0.6 mM to 0.9 mM, 0.7 mM to 0.9 mM, 0.8 mM to 0.9 mM, 0.5 mM to 0.8 mM, 0.6 mM to 0.8 mM, 0.7 mM to 0.8 mM, 0.5 mM to 0.8 mM, 0.6 mM to 0.8 mM, 0.5 mM to 0.7 mM, 0.6 mM to 0.7 mM, and 0.5 mM to 0.6 mM.

In embodiments, the concentration of DNase I is 20 U per μg of DNA template. In embodiments, the concentration of DNase I is selected from 1 U to 40 U/μg of DNA template, 1 U to 35 U/μg of DNA template, 1 U to 30 U/μg of DNA template, 1 U to 25 U/μg of DNA template, 1 U to 20 U/μg of DNA template, 1 U to 15 U/μg of DNA template, 1 U to 10 U/μg of DNA template, 5 U to 40 U/μg of DNA template, 5 U to 35 U/μg of DNA template, 5 U to 30 U/μg of DNA template, 5 U to 25 U/μg of DNA template, 5 U to 20 U/μg of DNA template, 5 U to 15 U/μg of DNA template, 5 U to 10 U/μg of DNA template, 10 U to 40 U/μg of DNA template, 10 U to 35 U/μg of DNA template, 10 U to 30 U/μg of DNA template, 10 U to 25 U/μg of DNA template, 10 U to 20 U/μg of DNA template, 10 U to 15 U/μg of DNA template, 15 U to 40 U/μg of DNA template, 15 U to 35 U/μg of DNA template, 15 U to 30 U/μg of DNA template, 15 U to 25 U/μg of DNA template, 15 U to 20 U/μg of DNA template, 20 U to 40 U/μg of DNA template, 20 U to 35 U/μg of DNA template, 20 U to 30 U/μg of DNA template, 20 U to 25 U/μg of DNA template, 25 U to 40 U/μg of DNA template, 25 U to 35 U/μg of DNA template, 25 U to 30 U/μg of DNA template, 30 U to 40 U/μg of DNA template, and 35 U to 40 U/μg of DNA template.

In embodiments, the DNase I incubation is performed at 37° C. for 10 min to 60 min, for example, from 10 min to 60 min, 10 min to 55 min, 10 min to 50 min, 10 min to 45 min, 10 min to 40 min, 10 min to 35 min, 10 min to 30 min, 10 min to 25 min, 10 min to 20 min, or 10 min to 55 min.

In embodiments, the incubation is performed at 37° C. for 10 min, 20 min, 30 min, 35 min, 40 min, 45 min, 50 min, 55 min, or 60 min.

Disclosed are materials, compositions, and components that can be used for, can be used in conjunction with, can be used in preparation for, or are products of the disclosed methods. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compounds. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

### EXAMPLES

### DNA Templates

Double-stranded DNA template encoding Firefly luciferase (Fluc) was generated by polymerase chain reaction. The DNA template included a T7 RNA polymerase promoter sequence, a 5′ untranslated region (5′UTR), open reading frame (ORF), a 3′ untranslated region (3′-UTR), and a poly(A) tail. Prior to its use in in vitro transcription (IVT) reactions, the DNA template was purified via column purification using a QIAGEN Plasmid Plus kit (Qiagen, Hilden, Germany) or a similar kit.

### 10×IVT Buffer 1 and Comparative Buffer

10×IVT Buffer 1 and Comparative Buffer were prepared using the concentrations for each component listed in Table 1.

TABLE 1
10X IVT Comparative Buffer and Buffer 1

| | 10X IVT | 10X IVT |
|---|---|---|
| | Buffer 1 | Comparative Buffer |
| Component | Concentration | Concentration |
| 1M Tris · HCl pH 7.5 | 400 mM | 400 mM |
| 12N HCl | 150 mM | N/A |
| 1M Mg⁺² | 160 mM, MgCl₂ | 165 mM, MgAc |
| 1M DTT | 100 mM | 100 mM |
| Spermidine | 20 mM | 20 mM |
| Triton X-100 | N/A | 0.02% |
| Final pH | ~1.5 (±0.1) | 8.0 |

### In Vitro Transcription

IVT reactions using 10×IVT Buffer 1 (Buffer 1 IVT reactions) and IVT reactions using 10×IVT Comparative Buffer (Comparative Buffer IVT reactions) comprising the components listed in Table 2 were prepared with Fluc DNA template.

The Comparative Buffer IVT reaction was prepared with 10×IVT Comparative Buffer (400 mM Tris-HCl (pH 8.0), 165 mM Magnesium acetate, 20 mM spermidine, 100 mM DTT, 0.02% Triton X-100), and incubated at 37° C. for 2-3 hours.

The Buffer 1 IVT reaction was prepared with 10×IVT Buffer 1 (400 mM Tris-HCl (pH 7.5), 150 mM HCl, 160 mM MgCl₂, 20 mM spermidine, 100 mM DTT), and incubated at 37° C. for 2-3 hours.

After 2 hours of incubation, optionally, Spike-in mix was added: each NTP (ATP, CTP, GTP, and UTP) was added for a final concentration of 4 mM (excludes NTPs already present in the reaction mixture), and 10×IVT reaction buffer was added to a final concentration of 1.74× reaction buffer (and ending with a 35% volume increase).

After the IVT incubation period (either 3 hours total or 2 hours followed by another 2 hours after Spike), the IVT reactions were supplemented with CaCl₂) (for a final concentration of 2 mM in the reaction mixture) and DNase I (20 U/μg of template DNA used; New England Biolabs; Ipswich, MA; Catalog No. M0303) and incubated for 20 minutes at 37° C. Comparative Buffer IVT reactions were supplemented DNase I reaction buffer and 0.30 U/μL DNase I (New England Biolabs; Catalog No. M0303) and incubated for 15 minutes at 37° C.

After DNase treatment, the resulting mRNA transcripts for each of the Comparative Buffer IVT reactions and Buffer 1 IVT reactions were purified using LiCl precipitation.

TABLE 2
IVT Comparison

| | | |
|---|---|---|
| | Comparative | |
| | Buffer IVT | Buffer 1 IVT |
| IVT SET UP | Final | Final |
| Component | Concentration | Concentration |

| | | | | |
|---|---|---|---|---|
| ATP (100 mM) | 5 | mM | 5 | mM |
| CTP (100 mM) | 5 | mM | 5 | mM |
| GTP (100 mM) | 5 | mM | 5 | mM |
| UTP (100 mM) | 5 | mM | 5 | mM |
| CleanCap analogs (100 mM) | 4 | mM | 10 | mM |

| | | |
|---|---|---|
| 10X Reaction Buffer | 1X, pH 8.0 | 1X, pH ~1.5 |
| DNA Templates (μg/μL) | 0.050 μg/μL or | 0.050 μg/μL or |
| | 0.025 μg/μL* | 0.025 μg/μL* |

| | | | | |
|---|---|---|---|---|
| RNase Inhibitor (40 U/μL) | 1 | U/μL | 1 | U/μL |
| iPP (0.1 U/μL) | 0.002 | U/μL | 0.002 | U/μL |
| T7 RNA Pol (50 U/μL) | 8 | U/μL | 22 | U/μL |
| (New England Biolabs) | | | | |

| | | |
|---|---|---|
| Triton X-100 | 0.002% | — |

*Final concentration of DNA template is 0.050 μg/μL for a plasmid template or 0.025 μg/μL for a PCR template.
**modified NTPs can be used in place of wild-type NTPs.

### Example 1: IVT mRNA Yield and Capping Efficiency of the mRNA Transcripts Synthesized Using m6A or 3′OMe m6A CleanCap Analogs

Buffer 1 IVT method was used to synthesize mRNA. In this example, firefly luciferase (Fluc) transcription template was used and two CleanCap analogs, m6A (^{m7}Gppp^{m6} A_{2′Ome}pG) or 3′OMe m6A (^{m7}G_{3′Ome}ppp^{m6}A_{2′Ome}pG), were used to cap the synthesized mRNAs.

The mRNA synthesis was carried out as described above, without the Spike-in step. The mRNA transcripts produced using Buffer 1 IVT conditions were purified by LiCl precipitation.

The mRNA capping efficiency was assessed by LC-MS analysis of cleaved 5′ mRNA fragments. The cleavage of mRNA samples was performed using 8-17 DNAzyme (5′ TTACTCTTCTTTTTCCGAGCCGGACGACTCTTATTTCCC 3′ (SEQ ID NO:1)) using a modified protocol (Silverman, et al. (2009). Use of deoxyribozymes in RNA research. Methods in Enzymology, 469, 95-117.) resulting in 13-mer 5′ fragments of mRNA. Post-cleavage samples were treated with T4 PNK and CIP enzymes to remove 3′ and 5′ phosphates, respectively, allowing for cleaner chromatograms and easier data interpretation in which any uncapped transcripts now contain a 5′ OH. Ion-Pairing Reverse Phase-High-Performance Liquid Chromatography/electrospray ionization tandem mass spectrometry (IP-RP-HPLC-ESI-MS) was performed on a Vanquish UHPLC (Thermo Fisher Scientific) equipped with a UV detector that was connected to a QExactive Orbitrap (Thermo Fisher Scientific). Mobile phase A included 1% hexafluoroisopropanol (HFIP), 0.1% N,N-diisopropylethylamine (DIEA), 2 μM ethylenediaminetetraacetic acid (EDTA), and mobile phase B included 0.075% HFIP 0.0375% DIEA, 2 μM EDTA in 65% acetonitrile (ACN). Analysis was performed with a Phenomenex Clarity Oligo-XT 2.1×50 mm, 2.6 μm column heated to 65° C. and a flow rate of 0.4 mL/min. The gradient profile for elution started at 2% B for 3 min, 0.1 min ramp to 6% B, linear ramp to 15% B over 19 min, linear ramp to 20% B for 11 min, 0.1 min ramp to 90% B for a 2 min rinse, finally a 0.1 min ramp 2% B for 6.7 min to re-equilibrate the column. All mass spectra were obtained in the negative ion mode, over a scan range of 800-2500 m/z. Spectra were analyzed using ProMass Deconvolution software (Novatia, Newton, PA) in high-resolution mode. The deconvoluted MS signal intensities (calculated by ProMass software for each individual species) corresponding to uncapped and capped cleaved 5′ mRNA fragments with correct observed masses based on calculated predictions were used in the calculating efficiency of mRNA capping.

The estimation of capping efficiency was made using the following formula: Capping efficiency, %=ΣMass Intensities of Identified 5′ capped species/(2 mass intensities of identified 5′ capped+5′ uncapped species)×100.

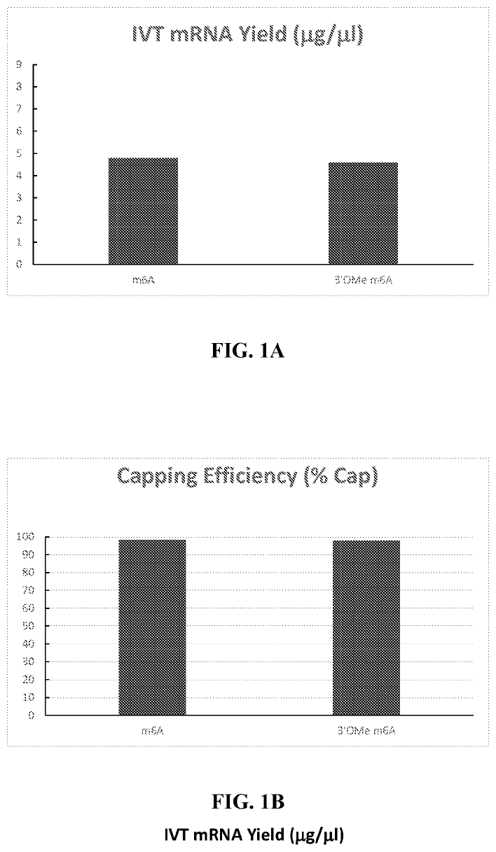

As shown in FIG. 1A, the Buffer 1 IVT method, with 3′OMe m6A (^{m7}G_{3′Ome}ppp^{m6} A_{2′Ome}pG), resulted in comparable mRNA transcript yields, compared to the Buffer 1 IVT method, with m6A (^{m7}Gppp^{m6} A_{2′Ome}pG). FIG. 1B shows that using the Buffer 1 IVT method with either m6A (^{m7}Gppp^{m6} A_{2′Ome}pG) or 3′OMe m6A (^{m7}G_{3′Ome}ppp^{m6} A_{2′Ome}pG) an mRNA capping efficiency of greater than 97% was achieved for Fluc transcripts.

### Example 2: Optimizing IVT mRNA Synthesis Conditions for 3′OMe m6A CleanCap Analog

Buffer 1 IVT method or Comparative Buffer IVT method was used to synthesize mRNA. In this example, firefly luciferase (Fluc) transcription template was used for transcription and CleanCap analog 3′OMe m6A (^{m7}G_{3′Ome}ppp^{m6} A_{2′Ome}pG) was used to cap the synthesized mRNAs.

The mRNA synthesis was carried out as described above. In some experiments there was no Spike-in step. The mRNA transcripts synthesized without the Spike-in step were called “Fixed”. In other experiments, the Spike-in step was added as described above. The mRNA transcripts synthesized with the Spike-in step were called “Spiked”. The Spike-in was carried out with NTPs and the same buffer as the buffer used during the first step of the mRNA synthesis.

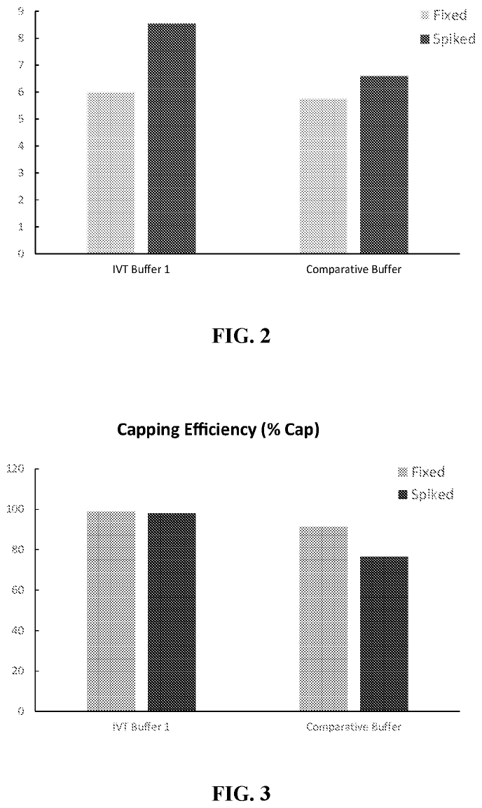

FIG. 2 shows that Buffer 1 IVT method produced higher yield of mRNA transcripts than Comparative Buffer IVT method, when there was no Spike-in step. Buffer 1 IVT method produced about 6 μg/μl mRNA transcripts and Comparative Buffer IVT method produced 5.77 μg/μl mRNA transcripts. When the Spike-in step was added, both methods produced more mRNA transcripts. Buffer 1 IVT method with Spike in step produced about 8.6 μg/μl mRNA transcripts and Comparative Buffer IVT method with Spike in step produced about 6.6 μg/μl mRNA transcripts.

Thus, Buffer 1 IVT method with Spike-in step is optimal for increased production of mRNA transcripts (FIG. 2).

FIG. 3 shows that capping efficiency is much higher (98.75%) when Buffer 1 IVT method was used compared to when Comparative Buffer IVT method was used (91.40%). Once the Spike-in step was added to the original IVT methods there was a large drop in capping efficiency when Comparative Buffer IVT method was used (76.49%), but not when Buffer 1 IVT method was used (97.94%).

Thus, Buffer 1 IVT method with or without Spike-in step is optimal for high capping efficiency (FIG. 3).

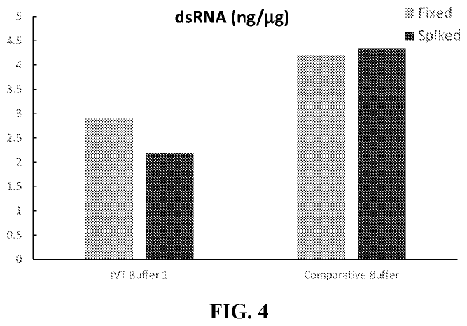

The Buffer 1 IVT method also resulted in mRNA transcripts with reduced immunogenicity, as evidenced by a decrease in double-stranded RNA (dsRNA) compared to the transcripts produced using the Comparative Buffer IVT method (FIG. 4). The amount of dsRNA was determined as described in Karico K. et al., (2011) Nucleic Acids Res. 39: e142. The mRNA transcripts produced were purified by LiCl precipitation.

In summary, as shown in Table 3, the mRNA transcripts produced using the Buffer 1 IVT method were equivalent to or better than mRNA transcripts produced using the Comparative Buffer IVT method when critical attributes were assessed. The Spike-in step improved the yield of the mRNA transcripts even further. Results in Table 3 were obtained with 3′OMe m6A (^{m7}G_{3′Ome}ppp^{m6} A_{2′Ome}pG) cap.

TABLE 3
Comparison of quality attributes for mRNA produced
using Comparative Buffer IVT method or Buffer
1 IVT method, with or without Spike-in step

| | Crude mRNA | | Capping | |
|---|---|---|---|---|
| Metric | Yield | | Efficiency | dsRNA |
| Buffer 1 IVT | 6 | μg/μL | 98.75% | 2.90 ng/μg |
| Buffer 1 IVT with Spike-in | 8.6 | μg/μL | 97.94% | 2.19 ng/μg |
| Comparative Buffer IVT | 5.77 | μg/μL | 91.40% | 4.22 ng/μg |
| Comparative Buffer IVT | 6.6 | μg/μL | 76.49% | 4.33 ng/μg |
| with Spike-in | | | | |

In summary, the Buffer 1 IVT method produces high yields of 5′ capped mRNA at high purity. Some IVT reactions yielded 9 g/L of 5′ capped mRNA, with less dsRNA impurities than other IVT methods. This method can be used in combination with purification processes to produce an mRNA product substantially free of prematurely aborted transcripts.

1. An in vitro method for synthesizing capped messenger RNA (mRNA) transcripts comprising:
(a) forming a reaction mixture comprising:
(i) 6.0 mM to 25 mM initiating capped oligonucleotide primer or a salt thereof,
(ii) 0.020 μg/μl to 0.1 μg/μl DNA template;
(iii) nucleoside triphosphates (NTPs), including 3 mM to 7 mM ATP; 3 mM to 7 mM CTP; 3 mM to 7 mM GTP; and 3 mM to 7 mM UTP;
(iv) 1× Transcription Buffer;
(v) 10 U/μL to 18 U/μL RNA polymerase; and
(vi) DNase and RNase free water,
wherein the reaction mixture has a pH of 6.0 to 8.0; and
(b) incubating the reaction mixture, for 2-3 hours, under conditions that allow transcription of the DNA template to obtain a solution comprising capped mRNA transcripts.

2. The method of claim 1, wherein the initiating capped oligonucleotide primer is a trinucleotide.

3. (canceled)

4. The method of claim 1, wherein the initiating capped oligonucleotide primer is ^{m7}Gppp^{m6}A_{2′Ome}pG or a salt thereof.

5. The method of claim 4, wherein ^{m7}Gppp^{m6} A_{2′Ome}pG has the following structure:

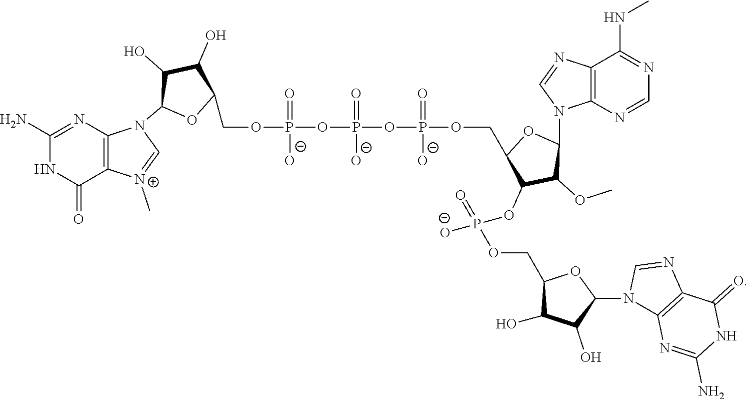

6. The method of claim 1, wherein the initiating capped oligonucleotide primer is ^{m7}G_{3′Ome}ppp^{m6} A_{2′Ome}pG or a salt thereof.

7. The method of claim 6, wherein ^{m7}G_{3′Ome}ppp^{m6}A_{2′Ome}pG has the following structure:

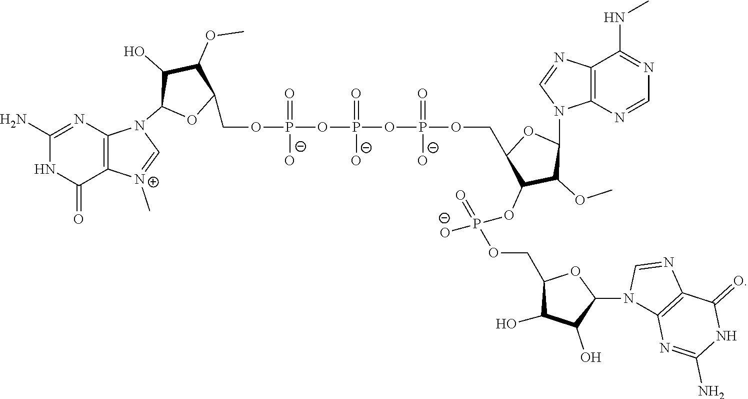

8. The method of claim 1, wherein one or more of the NTPs is a modified NTP.

9-26. (canceled)

27. The method of claim 1, wherein the 1× Transcription Buffer is a 1:10 dilution of a 10× Transcription Buffer, wherein the 10× Transcription Buffer comprises:
(a) about 400 mM Tris-HCl (pH 7.5);
(b) about 100 mM dithiothreitol (DTT);
(c) about 20 mM spermidine;
(d) about 160 mM magnesium chloride;
(e) about 150 mM hydrochloric acid;
(f) DNase and RNase free water; and
wherein the 10× Transcription Buffer has a pH of about 1.5.

28-31. (canceled)

32. The method of claim 1, further comprising:
(c) incubating the solution comprising capped mRNA transcripts with a Spike-in mix comprising:
(i) nucleoside triphosphates (NTPs), including ATP, CTP, GTP, and UTP, each with a concentration of 3 mM to 8 mM in a Final Reaction Mixture; and
(ii) 10× Transcription Buffer, with a concentration of 1× Transcription Buffer in a Final Reaction Mixture.

33-35. (canceled)

36. The method of claim 32, further comprising:
(d) incubating the solution comprising capped mRNA transcripts or the Final Reaction Mixture with:
(i) a solution of CaCl₂) with a concentration of 0.5 mM to 5 mM in the reaction mixture or the Final Reaction Mixture, and
(ii) DNase I.

37-46. (canceled)

47. The method of claim 1, wherein the method produces 7-13 mg/mL of mRNA transcripts, wherein greater than 95% of the mRNA transcripts are capped mRNA transcripts.

48-52. (canceled)

53. An in vitro method for synthesizing capped messenger RNA (mRNA) transcripts comprising:
(a) forming a reaction mixture comprising:
(i) 6.0 mM to 25 mM initiating capped oligonucleotide primer or a salt thereof;
(ii) 0.020 μg/μl to 0.1 μg/μl DNA template;
(iii) nucleoside triphosphates (NTPs), including 3 mM to 7 mM ATP; 3 mM to 7 mM CTP; 3 mM to 7 mM GTP; and 3 mM to 7 mM UTP;
(iv) 1× Transcription Buffer;
(v) 10 U/μL to 18 U/μL RNA polymerase; and
(vi) DNase and RNase free water,
wherein the reaction mixture has a pH of 6.0 to 8.0;
(b) incubating the reaction mixture, for 2-3 hours, under conditions that allow transcription of the DNA template to obtain a solution comprising capped mRNA transcripts;
(c) optionally incubating the solution comprising capped mRNA transcripts with a Spike-in mix:
(i) nucleoside triphosphates (NTPs), including ATP, CTP, GTP, and UTP, each with a concentration of 3 mM to 8 mM in a Final Reaction Mixture; and
(ii) 10× Transcription Buffer, with a concentration of 1× Transcription Buffer in a Final Reaction Mixture,
wherein the incubation is performed at 37° C. for 1 to 3 hours;
(d) incubating the solution comprising capped mRNA transcripts or the Final Reaction Mixture with:
(i) a solution of CaCl₂) with a concentration of 0.5 mM to 5 mM in the reaction mixture or the Final Reaction Mixture, and
(ii) DNase I,
wherein the incubation is performed at 37° C. for 10 to 60 minutes.

54-61. (canceled)

62. The method of claim 53, wherein the initiating capped oligonucleotide primer is ^{m7}Gppp^{m6} A_{2′Ome}pG or a salt thereof.

63. The method of claim 62, wherein ^{m7}Gppp^{m6}A_{2′Ome}pG has the following structure:

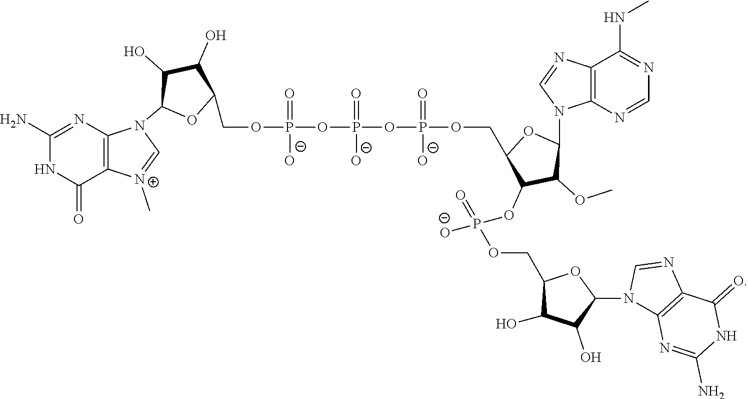

64. The method of claim 53, wherein the initiating capped oligonucleotide primer is ^{m7}G_{3′Ome}ppp^{m6} A_{2′Ome}pG or a salt thereof.

65. The method of claim 64, wherein ^{m7}G_{3′Ome}ppp^{m6} A_{2′Ome}pG has the following structure:

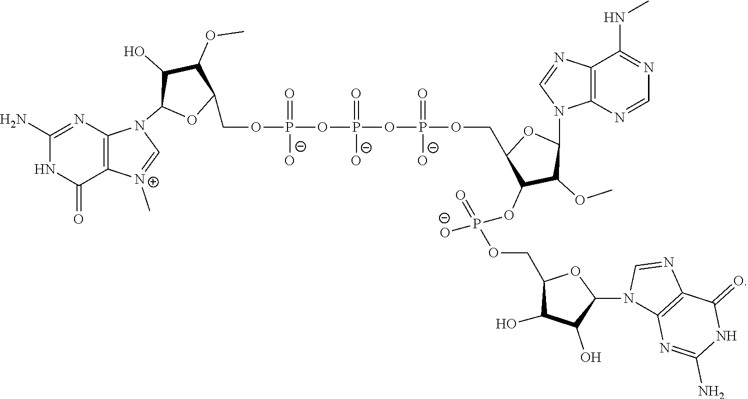

66-84. (canceled)

85. The method of claim 53, wherein the 1× Transcription Buffer is a 1:10 dilution of a 10× Transcription Buffer, and wherein the 10× Transcription Buffer comprises:
(a) about 400 mM Tris-HCl (pH 7.5);
(b) about 100 mM dithiothreitol (DTT);
(c) about 20 mM spermidine;
(d) about 160 mM magnesium chloride;
(e) about 150 mM hydrochloric acid;
(f) DNase and RNase free water; and
wherein the 10× Transcription Buffer has a pH of about 1.5.

86-95. (canceled)

96. The method of claim 53, wherein the method produces 7-13 mg/mL of mRNA transcripts, wherein greater than 95% of the synthesized mRNA transcripts are capped mRNA transcripts.

97-113. (canceled)
